(19) United States
(12) Patent Application Publication
(10) Pub. No.: US 2011/0229514 A1
(43) Pub. Date: Sep. 22, 2011

(54) VACCINE AND IMMUNIZATION METHOD USING PLASMODIUM ANTIGEN 2

(76) Inventors: Denise Doolan, Queensland (AU); Thomas L. Richie, Glenelg, MD (US); Robert Sauerwein, Amhem (NL)

(21) Appl. No.: 12/881,604

(22) Filed: Sep. 14, 2010

Related U.S. Application Data
(63) Continuation-in-part of application No. PCT/US09/01643, filed on Mar. 16, 2009.
(60) Provisional application No. 61/322,511, filed on Apr. 9, 2010.

(51) Int. Cl. A61K 39/015; A61P 33/06

(52) U.S. Cl. 424/199.1; 424/272.1

(57) ABSTRACT

A vaccine comprising an immunogenic preparation containing a plasmodium antigen or a fragment thereof, wherein said antigen is selected from a group consisting of PyAg2, PfAg2, PvAg2, PkAg2, PoAg2, PmAg2, PbAg2, PcAg2 and a combination thereof. A vaccination method comprising administering a priming immunization preparation containing a plasmodium antigen or fragment thereof, wherein said antigen is selected from a group consisting of PyAg2, PfAg2, PvAg2, PkAg2, PoAg2, PmAg2, PbAg2, PcAg2 and a combination thereof, and administrating a boosting immunization preparation containing said plasmodium antigen or fragment, wherein said antigen is selected from a group consisting of PyAg2, PfAg2, PvAg2, PkAg2, PoAg2, PmAg2, PbAg2, PcAg2 and a combination thereof. The vaccination method further comprising co-administration of an immune modulating drug.

50

### CROSS-REFERENCE TO RELATED APPLICATIONS

This application claims priority to provisional application No. 61/036,666 filed Mar. 14, 2008 and provisional application No. 61/322,511, filed Apr. 9, 2010, and is a continuation-in-part application of International Application No. PCT/US09/01643 filed Sep. 14, 2009.

### SEQUENCE LISTING

Incorporated by reference in its entirety herein is a paper copy of the nucleotide/amino acid sequence listing submitted concurrently herewith.

### BACKGROUND OF THE INVENTION

Malaria is one of the most devastating parasitic diseases affecting humans. Indeed, 41% of the world's population lives in areas where malaria is transmitted, including parts of Africa, Asia, the Middle East, Central and South America, Hispaniola, and Oceania. The World Health Organization (WHO) and the Centers for Disease Control (CDC) estimate that malaria infects 300-500 million people and kills 700,000-3 million people annually, with the majority of deaths occurring in children in sub-Saharan Africa. Malaria also is a major health concern to U.S. military personnel deployed to tropical regions of the world. For example, in August 2003, 28% of the 26^{th} Marine Expeditionary Unit and Joint Task Force briefly deployed to Monrovia, Liberia, were infected with the malaria parasite Plasmodium falciparum. In addition, one 157-man Marine Expeditionary Unit sustained a 44% malaria casualty rate over a 12-day period while stationed at Robert International Airport in Monrovia. In all conflicts during the past century conducted in malaria endemic areas, malaria has been the leading cause of casualties, exceeding enemy-inflicted casualties in its impact on “person-days” lost from duty.

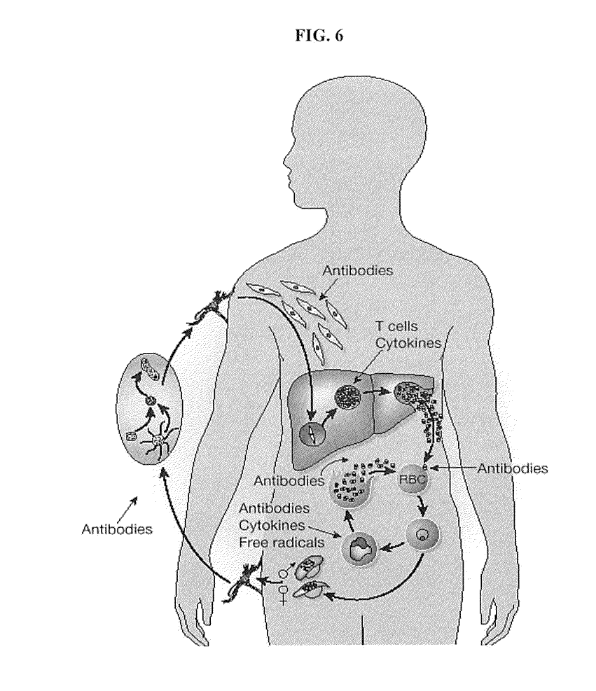

Malarial parasite infects both mammalian and mosquito hosts during its complex life cycle (see FIG. 6) and is caused by a protozoan parasite, which has a complex multi-stage lifecycle involving both intra- and extracellular stages in human host and mosquito vector. Malaria infection starts by the bite of an infected Anopheles mosquito that inoculates sporozoite forms. Via the bloodstream they reach the liver and invade, mature and multiply in hepatocytes. Once released by infected hepatocytes into the bloodstream as pathogenic asexual forms they start to multiply in invaded red blood cells. A small fraction of blood stage parasites are committed to become sexual forms and mature into gametocytes which are responsible for transmission to mosquitoes. Mature gametocytes are ingested by blood-feeding mosquitoes and differentiate after a number of transitions into sporozoites and migrate to the salivary gland. At each mosquito bloodmeal these motile parasites are injected into the human host resulting in the spread of the parasite and associated disease in the population. Unlike sporozoites and gametocytes which are clinically silent, only asexual stages of the life cycle are responsible for clinical symptoms, complications and the possibility of death.

To combat malaria during U.S. military operations, preventive drugs, insect repellants, and barriers have been used with some success, but developing drug resistance by the malaria parasite and insecticide resistance by mosquito vectors has limited the efficacy of these agents. Moreover, the logistical burden and side effects associated with the use of these agents often is associated with high non-compliance rates. Vaccines are the most cost effective and efficient therapeutic interventions for infectious diseases. In this regard, vaccination has the advantage of administration prior to military deployment and likely reduction in non-compliance risks. However, decades of research and development directed to a malaria vaccine have not proven successful.

The field of malaria vaccinology has followed the traditional vaccine approach: expose the host to a malaria antigen or antigens and, in the case of subunit vaccines, maximally stimulate the immune response using adjuvants, immune-stimulatory compounds or self-adjuvanting delivery systems such as viral vectors or virus-like particles. No vaccine based on this paradigm has worked well, however, for inducing high grade protection against malaria in humans. The majority of candidates have failed. Different formulations of a number of antigens have been tested in Phase 1 trials, and only about a dozen candidates have been evaluated in Phase 2 clinical field trials (WHO. Date Accessed. Malaria Vaccine Rainbow Tables; URL: http://www.who.int/vaccine_research/links/Rainbow/en/index.html). Approximately 40 trials were reported as of 1998 (see Engers and Godal, Parisitology Today, 14: 56-64 (1998)). Most of these trials have been directed against the sporozoite stage or liver stage of the Plasmodium life cycle, where the use of experimental mosquito challenges allows rapid progress through Phase 1 to Phase 2a preliminary efficacy studies. Anti-sporozoite vaccines tested include completely synthetic peptides, conjugates of synthetic peptide with proteins such as tetanus toxoid (to provide T cell help), recombinant malaria proteins, particle-forming recombinant chimeric constructs, recombinant viruses, and bacteria and DNA vaccines. Several trials of asexual blood stage vaccines have used either synthetic peptide conjugates or recombinant proteins. There also have been trials of transmission blocking vaccines (e.g., recombinant Pfs25).

The best vaccine to date, RTS,S, does not provide long term protection against infection but delays patency and reduces clinical severity. After several field trials demonstrating roughly 50% protection as measured by delays in the time to acquiring parasitemia or clinical malaria, RTS,S is now undergoing testing in a Phase 3 multicenter trial in Africa [7;8]. Recent efforts have focused on developing vaccines against several specific malaria genes and delivery vector systems including adenovirus, poxvirus, and plasmids. The current status of malaria vaccine development and clinical trials is reviewed in, for example, Graves and Gelband, Cochrane Database Syst. Rev., 1: CD000129 (2003), Moore et al., Lancet Infect. Dis., 2: 737-743 (2002), Carvalho et al., Scand. J. Immunol., 56: 327-343 (2002), Moorthy and Hill, Br. Med. Bull., 62: 59-72 (2002), Greenwood and Alonso, Chem. Immunol., 80: 366-395 (2002), and Richie and Saul, Nature, 415: 694-701 (2002).

When the parasite changes hosts, it develops motile stages that can invade the target organ in the new host, heading for the site where it can proliferate to the next motile stage. In this migration, the parasites encounters host cell barriers that hamper its advance. To circumvent these barriers and penetrate the organ, the parasite invades and traverses host cells. Cell-traversal ability is thus essential for the parasite to establish an infection in a new host.

Because motile stages of malarial parasite have no locomotory organelles, such as flagella or cilia, their motility requires substrates and is achieved by highly developed cytoplasmic secretary organelles, called micronemes. A secretory microneme protein, named cell-traversal protein of Plasmodium ookinetes and sporozoites (CelTOS or antigen 2, Kariu et al 2006) was found to have crucial role in the parasite invasive motility.

Targeted disruption of the CelTOS gene in Plasmodium berghei reduced parasite infectivity in the mosquito host approximately 200-fold. The disruption also reduced the sporozoite infectivity in the liver and almost abolished its cell-passage ability. Liver infectivity was restored in Kupffer cell-depleted rats, indicating that CelTOS is necessary for sporozoite passage from the circulatory system to hepatocytes through the liver sinusoidal cell layer. Electron microscopic analysis revealed that celtos-disrupted ookinetes invade the midgut epithelial cell by rupturing the cell membrane, but then fail to cross the cell, indicating that CelTOS is necessary for migration through the cytoplasm. These results suggest that conserved cell-passage mechanisms are used by both sporozoites and ookinetes to breach host cellular barriers.

In the instant application, antigen 2 (same as “CelTOS”, hereafter “Antigen 2” or “Ag2”) is first identified as one of malarial vaccine candidate from the genomic sequences of complex pathogens via an antigen identification strategy, which integrates bioinformaic predictions, HLA-supertype consideration, and in vitro cellular assays. Antigen 2 has also been shown to be highly expressed in the P. falciparum sporozoite/liver proteome as evidenced by MudPIT of P. falciparum sporozoites and P. falciparum sporozoite gene transcript profiles. In immune screening studies, Ag2 is recognized by volunteers either experimentally immunized with radiation-attenuated P. falciparum sporozoites or naturally exposed to malaria, and is preferentially recognized by irradiated sporozoite immunized volunteers who were protected against parasite challenge as compared to unprotected volunteers. Furthermore, animal data indicates that Ag2 is protective against P. yoelii challenge.

Malaria parasites generate strong immune responses, and a degree of protective immunity can be acquired through natural exposure, although the mechanisms of protection are poorly understood [1, 2]. The development of this immunity is marked initially by the ability to control the clinical symptoms associated with parasitemia, allowing the individual to tolerate significant parasite densities without getting the disease. The type of clinical immunity, typically developing in children, is followed by resistance to parasitemia, such that older children and adults no longer experience high densities of asexual forms in the blood. However, sterile immunity is never observed in naturally exposed populations; adults living in endemic areas often harbor parasites albeit at low densities, and will promptly re-acquire infections if cured through the administration of anti-malarial drugs. This clinical immunity is difficult to acquire and wanes once exposure is withheld.

Where and how invading Plasmodia are recognized and processed by the immune system is not well understood, but likely involves dendritic cells [DCs] with extracellular and intracellular pattern recognition receptors [PRRs]. PRR signal transduction determines the nature of the DC response, and is modulated by the PRRs involved, antigen dose, duration of exposure and microenvironment. Both TLR-dependent [TLR2, TLR4 and TLR9] as well TLR-independent [NALP3 inflammasome] pathways are involved in Plasmodium recognition but major differences between human and murine immune systems hamper conclusive interpretation [10-13]. Activation of DCs preferentially leads to their maturation followed by induction of T effector cells or it may lead to tolerogenic responses by induction of regulatory T cells [Tregs]. The ratio of effector and regulatory responses may influence the risk of productive malaria infection and clinical disease [14]. Protection is accomplished by stage-specific host effector responses and seems to require both cellular and humoral components. While specific antibodies are primarily important against sporozoites and blood stages, distinct cellular responses are required for protection against liver stage parasites [2].

Previously it has been shown that radiation-attenuated sporozoites induce >90% protection in humans [4]. Irradiation of infectious mosquitoes disrupts the gene expression of sporozoites, which remain capable of hepatocyte invasion but are no longer capable of complete liver-stage maturation or progression to the pathogenic blood stage. However, this generally requires 1000 bites by irradiated mosquitoes during five or more immunization sessions, which is a strikingly 20-fold lower potency than the 45 bites in the human model using chloroquine. Similar results are found in mouse studies [23].

Low parasitemia may induce protective effector mechanisms as shown by Pombo et al. where repeated intravenous injections of low numbers of parasites followed by a curative treatment before patency resulted in protection against a subsequent blood stage challenge administered without curative drugs [3]. More recently, it was shown in a murine model that subpatent blood stage infection with genetically attenuated blood stage parasites likewise provides complete protection apparently through both humoral and cellular immune responses [24]. In contrast, high parasitemia as observed in the field may inhibit the development of protective immunity and may relate to inhibition of cross-presentation required for the induction of cytotoxic T cells [18-20]. A cost-effective vaccine requires efficient induction of protective immunity over a short period of time and should therefore perform better than nature.

In the current inventive method, an embodiment is to combine the administration of a traditional vaccine with the separate administration of a drug designed to modulate the immune response to the vaccine thereby enhancing the potency or duration of the protective immune response. The drug may be administered once or multiple times, and the administration can be prior to, concurrent with, or following the administration of the vaccine (or a combination of these). The drug can be any drug that affects the immune response of the vaccine recipient and that is found empirically to enhance the protective effects of the vaccine. For example, it may be an immunostimulatory drug that improves antigen processing and presentation, or an immunosuppressive drug that curtails unwanted immune responses to vaccine antigens that may otherwise play a decoy function.

In a preferred embodiment, co-administration of chloroquine may enhance protection induced by sporozoite inoculation due to lowering of parasitemia and/or its immune modulating effects. Short-course treatment of mice with chloroquine improves the priming of naïve CD8+ T cell responses against soluble antigens in vivo [30]. Cross-presentation of soluble viral antigens to specific CD8+ T cell clones in vitro is improved when DCs are pulsed with the antigen in the presence of chloroquine. Furthermore, if hepatitis B virus vaccine [HBV] responders are further boosted together with a single dose of chloroquine a substantial increase of HBV-specific CD8+ T cells is observed [31]. Presentation of soluble viral antigens to specific CD8+ T cell clones by DCs is greatly improved in the presence of chloroquine, which prevents endosomal acidification, and seems to promote the transfer of endocytosed material into the cytosol. The net effect will depend on the routing and processing conditions such as acidification of the endosomal compartment [32].

There may be more drug choices to explore for co-administration during vaccination. Chemotherapeutic drugs have shown potential benefits for the immune response against tumors [33,34]. It has for instance been shown that low dose cyclophosphamide selectively depletes Tregs in both animal models and cancer patients with resulting enhancement of T cell functions. The potential benefits and safety risks of low dose cytostatic drugs in healthy volunteers should be carefully considered. The combined data show that orchestration of the antigen presenting pathways by drug modulation might tailor the immune response to a desired profile.

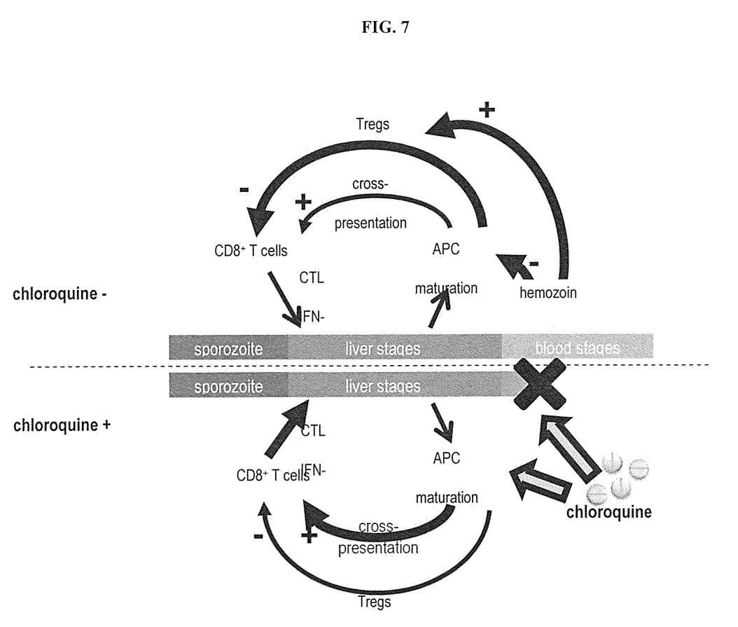

The functionality of the increased numbers of Tregs observed in human and animal malaria is not clear but there is an inverse correlation during acute disease between Tregs and malaria-specific memory responses [35]. Antigen presentation by immature or partially mature DCs conditions the emergence of Tregs. To activate Tregs, TLR9 signaling in dendritic cells is required and mediated by hemozoin, a digestion product of hemoglobin produced by Plasmodium that is involved in TLR9 activation [11]. Since chloroquine abrogates hemozoin-mediated cytokine production, this drug might inhibit this evading mechanism leading to a more effective establishment of immunological memory [36] [FIG. 7].

Given the long half-life of chloroquine, a single administration might maintain effective plasma levels throughout the induction phase of an immune response, making it possible to administer single doses of co-drug and vaccine as the immunization procedure. In the RUNMC study, the vaccine component consisted of intact P. falciparum sporozoites, presenting a potentially insurmountable safety concern [5].

A first step would be to test these combinations in order to determine the minimal protective dose of both vaccine and co-drug, as well as the fewest number of immunizations needed to achieve high grade protection. If chloroquine or another antimalarial were chosen, this approach could provide an added level of safety since it would treat any emergent blood stage infections even if the sporozoite vaccine were only partially attenuated. As long as the vaccine strain was completely sensitive to the co-drug, both immune modulation and protection against breakthrough blood stage infections could be achieved at the same time. Practical application will depend on the selected drug, drug half-life and vaccination regime.

### DETAILED DESCRIPTION OF THE FIGURES

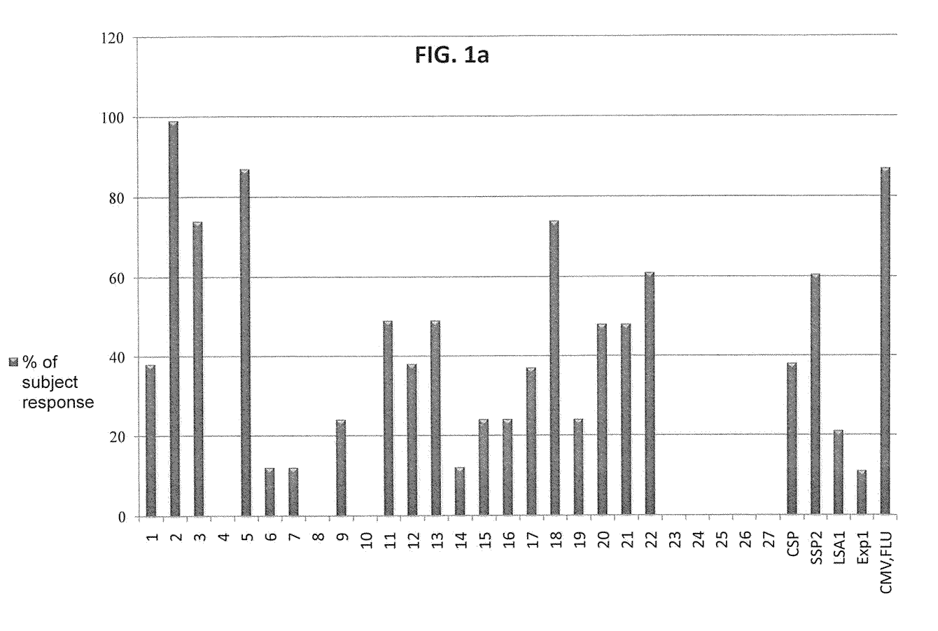

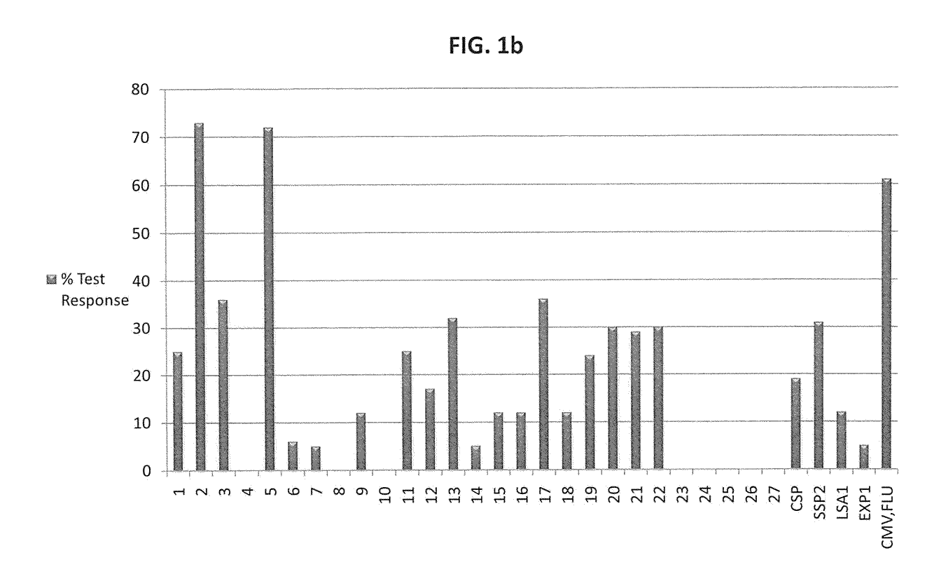

FIG. 1A. Antigen-specific reactivity in irradiated sporozoite-immunized volunteers—subject response rate.

FIG. 1B. Antigen-specific reactivity in irradiated sporozoite-immunized volunteers—test response rate.

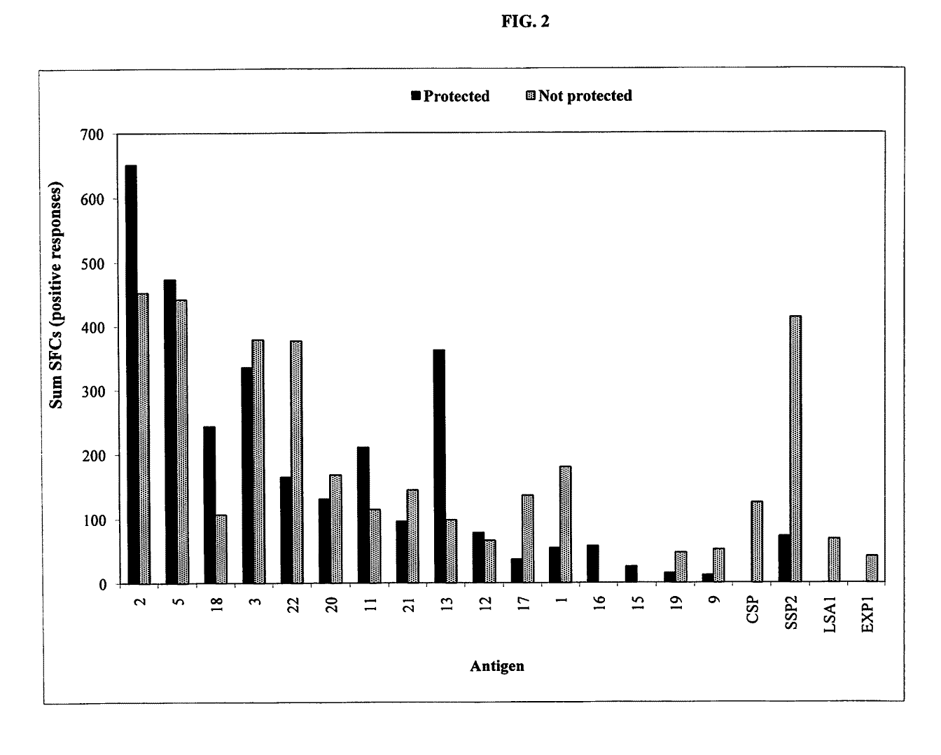

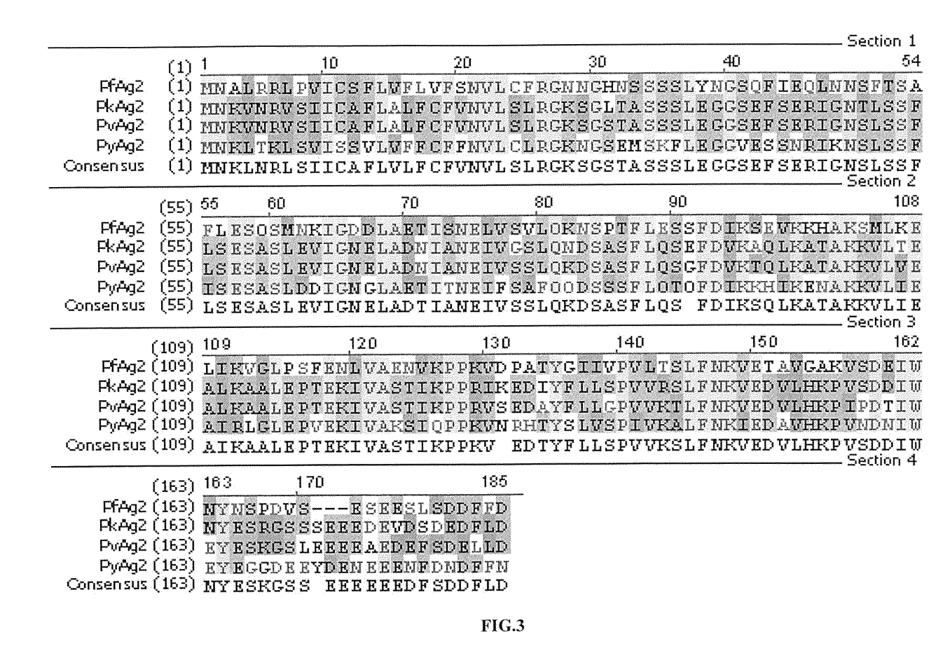

FIG. 2. Antigen-specific reactivity in irradiated sporozoite-immunized volunteers protected or non-protected against sporozoite challenge.

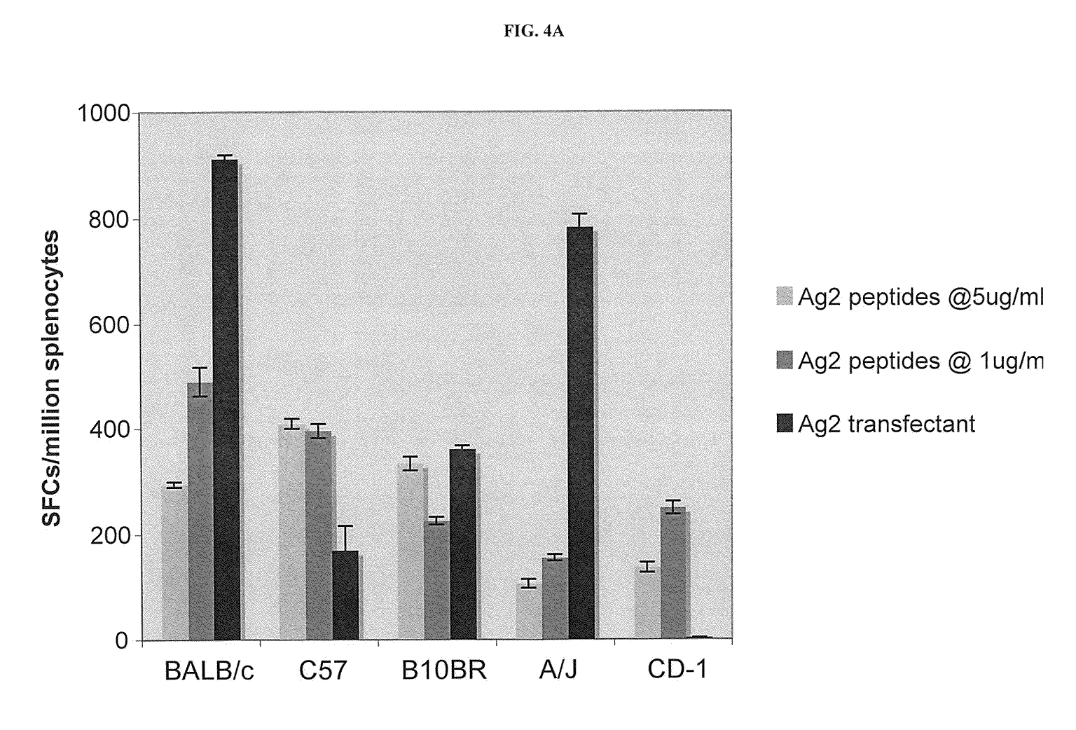

FIG. 3. Alignment of amino acid sequences of P. falciparum, P. vivax, P. knowlesi and P. voelii Ag2 orthologues.

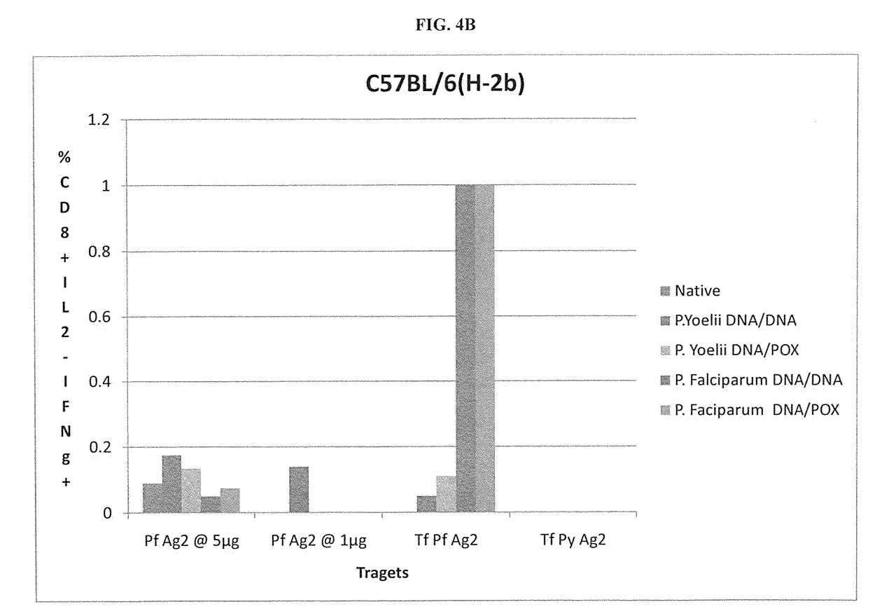

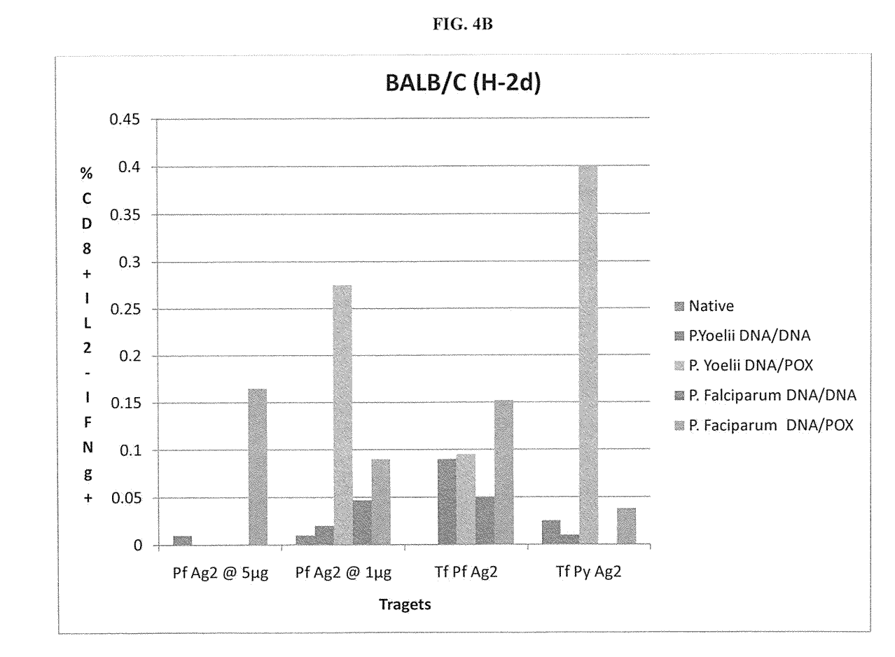

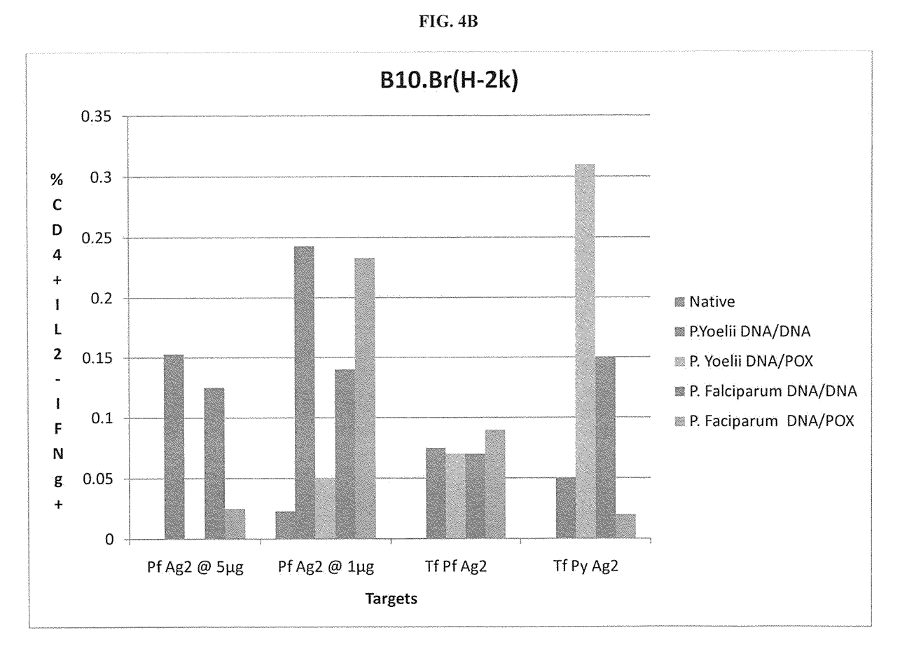

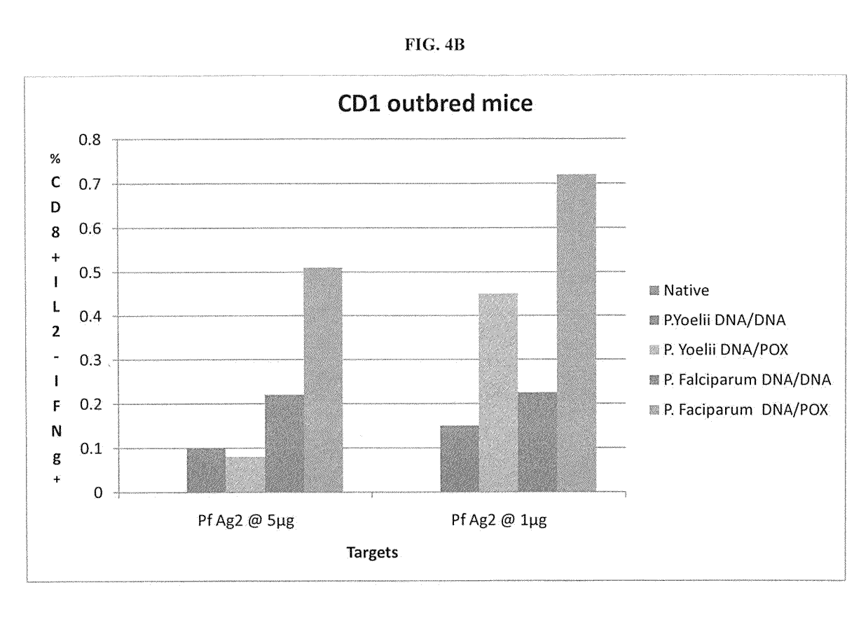

FIG. 4A. IFN-g ELIspot of PfAg2 DNA/DNA in immunized inbred and outbred mice.

FIG. 4B. Intracellular cytokine staining (ICS) of PfAg2 and PyAg2 DNA/DNA or DNA/PDX immunized inbred.

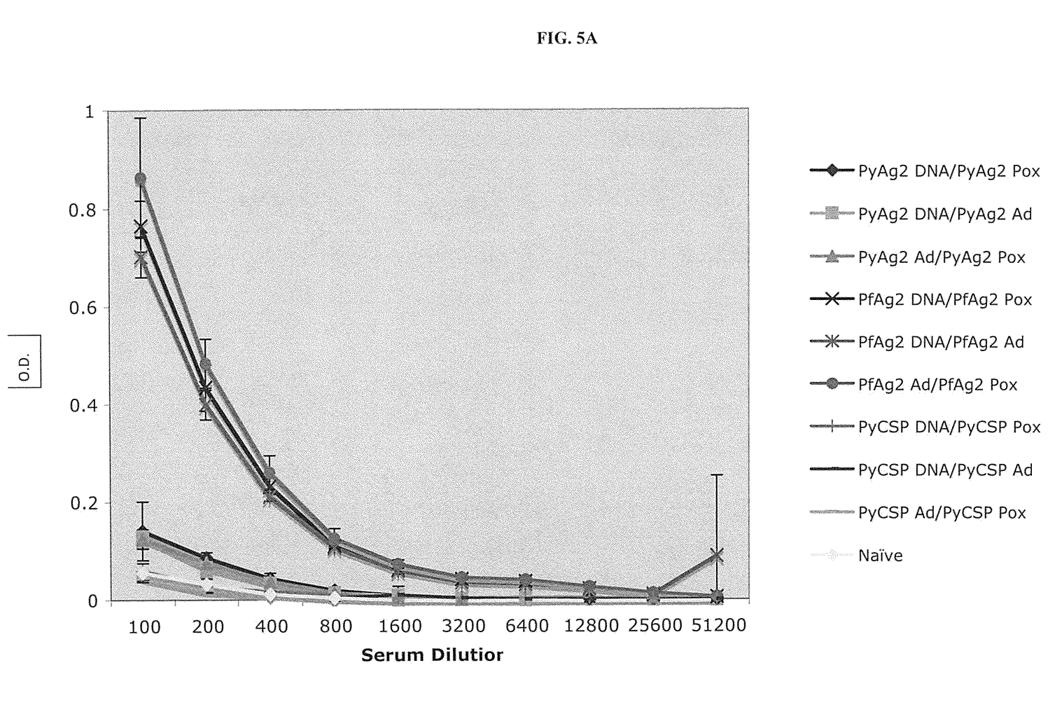

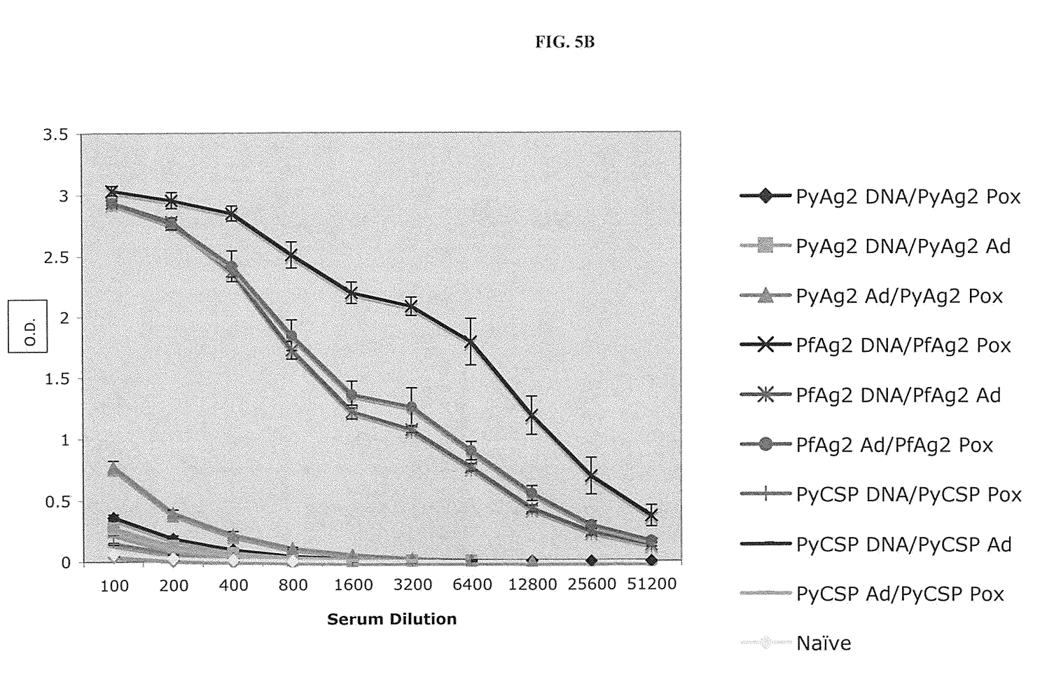

FIG. 5. Ag2 enzyme-linked immunosorbent assay (ELISA) against recombinant Ag2 protein. (A) Pre-boost sera. (B) Post-boost/Pre-challenge sera.

FIG. 6. Plasmodium lifecycle.

FIG. 7. Model for modulation of cellular responses by chloroquine during malaria. Blood stage parasites inhibit the development of liver stage immunity via antigen presenting cells and hemozoin. This results in upregulation of Tregs and therefore less effective establishment of CD8+ T cell responses. Chloroquine prevents the development of blood stage parasitemia, thereby diminishing the immune-evasing action of these parasites. Furthermore, it inhibits Treg induction and improves crosspresentation, therefore leading to a more effective CD8+ T cell response.

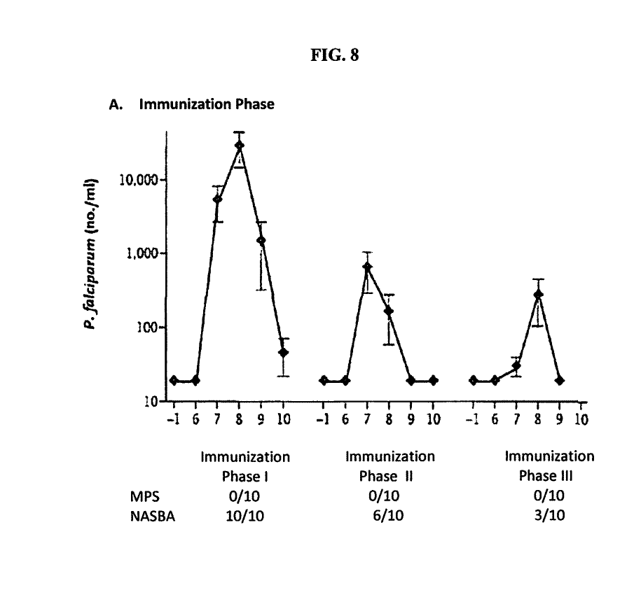

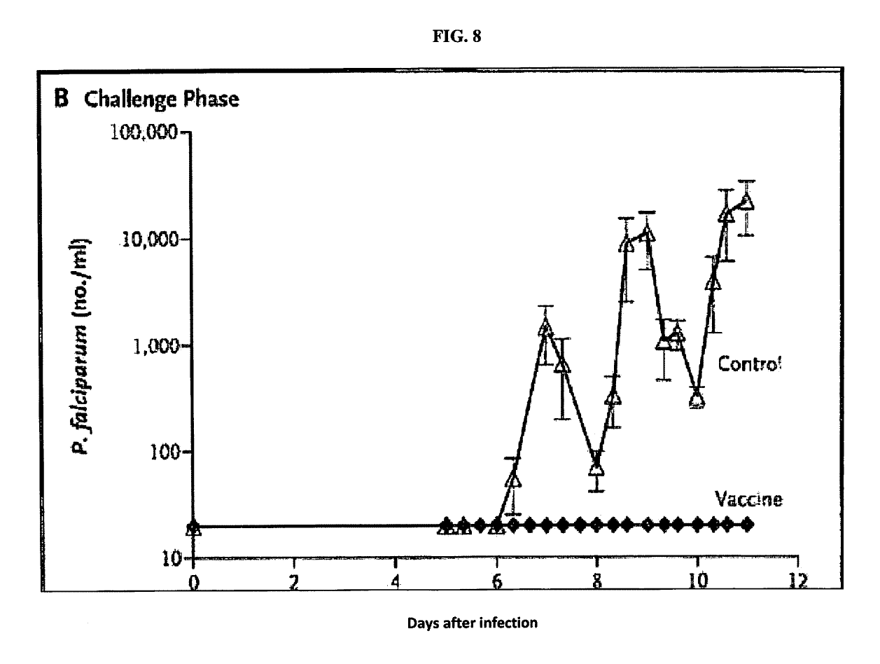

FIG. 8. Panel A shows the mean number of Plasmodium falciparum parasites per milliliter, as measured by nucleic acid sequence-based amplification (NASBA), after each of three immunizations on days before infection and during expected blood-stage parasitemia (days 6 to 10) in the vaccine group. The numbers of subjects who had positive results on peripheral-blood smears for malarial parasites (MPS) and NASBA are shown below the graph. Panel B shows the mean number of P. falciparum parasites in 5 subjects in the control group and 10 subjects in the vaccine group, as determined by real-time polymerase-chain-reaction assay before treatment with artemether-lumefantrine. The I bars denote standard errors.

FIG. 9. The proportion of lymphocytes that produced interferon-γ(IFN-γ) and interleukin-2 (Panel A), tumor necrosis factor a (TNF-α) and interleukin-2 (Panel B), or IFN-γ, TNF-α, and interleukin-2 (Panel C) after in vitro stimulation with erythrocytes infected with the homologous strain of P. falciparum (PfRBC), with uninfected erythrocytes (uRBC), or with phytohemagglutinin (PHA) as a positive control are shown before immunization (day I-1) and before malaria challenge (day C-1). Dashed lines represent the proportion of positive cells in unstimulated wells (culture medium only). The geometric mean fluorescence intensity of cells producing IFN-γ (Panel D), TNF-α (Panel E), and interleukin-2 (Panel F) that were isolated from vaccinees on day C-1 is shown after stimulation in vitro with infected erythrocytes. Cells are grouped according to their positivity or negativity for each of the other two cytokines. In Panels G, H, and I, the proportion of lymphocytes that produced IFN-γ and interleukin-2 in response to infected erythrocytes are shown on day I-1 and day C-1 for lymphocyte phenotypes, including naive T cells (CD3+CD45RO−), memory T cells CD3+CD45RO+), and non-T lymphocytes (CD3−CD45RO−) (Panel G); for T-cell phenotypes, including helper T cells (CD4+CD8−), cytotoxic T cells (CD4−CD8+), and other lymphocytes (CD4−CD8−) (Panel H); and for memory phenotypes, including naive T cells (CD62L+CD45RO−), central memory T cells (CD62L+CD45RO+), effector memory T cells (CD62L−CD45RO+), and other lymphocytes (CD62L−CD45RO−) (Panel I). The proportions of lymphocytes that produced IFN-γ and interleukin-2 after stimulation with uninfected erythrocytes were below 0.005% (not shown). All P values are for the comparison between the vaccine group and the control group and were calculated with the use of the Mann-Whitney test. The T bars represent standard errors.

### DETAILED DESCRIPTION OF THE INVENTION

### Definition

As used herein, the term “antigen” means an immunogenic peptide or protein which induces an immune response (see below) to a malarial pathogen capable of infecting a mammal.

The term “immune response” or “immunization” refer to the development in a subject of a humoral and/or cellular immunological response to an antigen that has been administered to the subject by the methods of this invention. “Humoral” immune responses refer to the production of antibodies, and a “cellular” immune response refers to the activation of T-lymphocytes, particularly cytolytic T-cells (“CTLs”) and helper T-cells. Specific T-cells involved in the cellular immune response include CD4+ and CD8+ T-cells.

To stimulate the humoral arm of the immune system, i.e. the production of antigen-specific antibodies, an “immunogenic fragment” will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, that define an epitope, or any integer between 5 amino acids and the full-length sequence, provided that the fragment in question retains immunogenic activity, as measured by an assay, such as the ones described herein.

Regions of a given polypeptide that include an “epitope” can be identified using any number of epitope mapping techniques, well known in the art. (See, e.g., Epitope Mapping Protocols in Methods in Molecular Biology, Vol. 66, Glenn E. Morris, Ed., 1996, Humana Press, Totowa, N.J.). Generally, T-cell epitopes which are involved in stimulating the cellular arm of the subject's immune system, are short peptides of 8-25 amino acids, and these are not typically predicted by the above-described methods for identifying humoral epitopes. A common way to identify T-cell epitopes is to use overlapping synthetic peptides and analyze pools of these peptides, or the individual ones, that are recognized by T cells from animals that are immune to the antigen of interest, using an enzyme-linked immunospot assay (ELISPOT). These overlapping peptides can also be used in other assays such as the stimulation of cytokine release or secretion, or by the ability to interact with major histocompatibility (MHC) tetramers. Such immunogenic fragments can also be identified based on their ability to stimulate lymphocyte proliferation in response to stimulation by various fragments from the antigen of interest. The term “epitope” as used herein refers to a sequence of at least about 3 to 5, preferably about 5 to 10 or 15, and not more than about 1,000 amino acids (or any integer therebetween), which define a sequence that by itself or as part of a larger sequence, binds to an antibody generated in response to such sequence or stimulates a cellular immune response. There is no critical upper limit to the length of the immunogenic fragment, which may comprise nearly the full-length of the protein sequence, or even a fusion protein comprising two or more epitopes from a single or multiple malarial parasite proteins. An epitope for use in the subject invention is not limited to a polypeptide having the exact sequence of the portion of the parent protein from which it is derived. Indeed, there are many known species of Plasmodium and the parasite retains the ability to continue to adapt, and there are several variable domains in the parasite that exhibit relatively high degrees of variability between species. Thus the term “epitope” encompasses sequences identical to the native sequence, as well as modifications to the native sequence, such as deletions, additions and substitutions (generally conservative in nature).

One component of the methods of the present invention is the use of a “priming” immunization, comprising the initial administration of one or more antigens to an animal, especially a human patient, in preparation for subsequent administration(s) of the same antigen. Specifically, the term “priming”, as used herein, defines a first immunization using an antigen which induces an immune response to the desired antigen and recalls a higher level of immune response to the desired antigen upon subsequent re-immunization with the same antigen when administered in the context of the same or a different vaccine delivery system. Specifically as used in this application, a “priming immunization” refers to the administration of a composition comprising a preparation containing a malarial antigen. As used herein, a “priming immunogenic composition or preparation” refers to a preparation containing a malarial antigen or fragment thereof with the preparation being selected from the group consisting of a recombinant virus expression system; a recombinant protein antigen or a recombinant polypeptide; a synthetic peptide; a polynucleotide vector; a whole organism or extract and combinations thereof.

Another component of the methods and compositions of the present invention is the use of a “boosting immunization”, or a “boost”, which means the administration of a composition delivering the same malarial antigen as encoded in the priming immunization, but often utilizing a preparation with different platform, such as those selected from the group consisting of: a recombinant virus expression system; a recombinant protein antigen or a recombinant polypeptide; a synthetic peptide; a polynucleotide vector; a whole organism or extract and combinations thereof. A boost is sometimes referred to as an anamnestic response, i.e. an immune response in a previously sensitized animal. A boosting immunization or a boosting immunogenic composition can comprise multiple doses, which may be the same or different amounts. As used in this application, a boosting immunization or a boosting immunogenic composition can comprise one, two, three or multiple doses.

The inventive subject matter relates to a method for inducing immune response and protective immunity in humans and animals using antigen 2 or CelTOS or a fragment thereof comprising immunizing with a priming immunization preparation that selected from a group consisting of recombinant virus expression system (such as recombinant poxvirus or recombinant adenovius); a recombinant protein antigen or a recombinant polypeptide; a synthetic peptide; a DNA vector; a whole organism or extract thereof; and a combination thereof, and subsequently immunizing with a boosting immunization preparation that is selected from a group consisting of a recombinant virus expression system such as recombinant poxvirus and recombinant adenovius; a recombinant protein antigen or a recombinant polypeptide; a synthetic peptide; a DNA vector; a whole organism or extract thereof; and a combination thereof. Antigen 2 is selected from the group consisting of PfAg2 (P. falciparum), PyAg2 (P. yoelii), PvAg2 (P. vivax), PkAg2 (P. knowlesi), PoAg2 (P. ovale), PmAg2 (P. malariae) PbAg2 (P. Berghei), PcAg2 (P. Chabaudi), and combination thereof. The immunization preparation may comprise plasmodium antigen 2, its fragment, or one or moe epitope of antigen 2, or a combination thereof. The suitable recombinant virus expression system may be used include poxvirus, adenovirus, adeno-associated virus, and retrovirus. Poxvirus used may be cowpox, canarypox, monkeypox, and fowlpox. In an embodiment of the inventive method, the immunization preparation may be administered with 1-4 priming dose, and followed by 1-4 boosting dose. The immunization preparations are administered by routes selected from the group consisting of subcutaneous, intramuscular, intradermal, mucosal, oral, transcutaneous, and by injection devices, or a via a combination of routs.

The invention further relates to a method of counteracting the immune modulating effects of the parasite that result in slow or partial induction of protection and effective memory responses. This is achieved by co-administration of drugs with immune modulating properties.

### EXAMPLE 1

### Genomic and Proteomic Identifications of Malaria Vaccine Candidate

The ImmunoSense strategy (Doolan et al. 2003) was designed to identify target antigens and maps T cell epitopes from large and complex genomes, such as P. falciparum, by integrating genomic and proteomic data with bioinformatic predictions, HLA supertype considerations, high-throughput binding assays, and cellular assays. A central component of the strategy is the capacity of the antigen to be recognized by immune responses thought to contribute in protection, allowing for rational antigen selection and prioritization.

T cells recognize a complex between a specific MHC type and a particular pathogen-derived linear T cell epitope, and a given epitope will thus elicit a response only in individuals who express an MHC molecule capable of binding that epitope. Human MHC molecules are extremely polymorphic, and different HLA types are expressed at dramatically different frequencies in different ethnicities. One means of circumventing the problem of genetic restriction relies on the selection of epitopes restricted by HLA types that can be grouped in broad families, called HLA supertypes, which are characterized by largely overlapping peptide repertoires and are expressed at high frequencies in all major ethnicities (Sette and Sidney 1999). By targeting HLA-A1, -A2, -A3/A11, -A24, -B7 and -B44 supertypes, coverage of virtually 100% of all populations irrespective of the ethnicity of the target population can be achieved.

In proof-of-concept studies (Doolan et al. 1993), starting with 27 ORFs defined by multidimensional protein identification technology (Florens et al. 2002), 16 novel proteins were identified and reproducibly recognized by at least two of the eight irradiated sporozoite immunized volunteers tested in two or more assays, but not by any of the four mock immunized controls. Nine antigens were highly antigenic (recognized by at least 50% of irradiated sporozoite volunteers in at least 25% of assays) three antigens were of intermediate reactivity (recognized by at least 25% of volunteers in at least 15% of assays), and four were of low reactivity (recognized by at least 10% volunteers in at least 5% of assays). Only one protein (Ag2) was recognized by all 8 volunteers. In comparison, CSP was recognized by 3 of 8 volunteers, SSP2 by 5 of 8, LSAT by 2 of 8, and Exp1 by 1 of 8 volunteers (Table 1).

TABLE 1
Summary of immune reactivities against the panel of
27 putative and four known P. falciparum antigens.

| | | # positive | % positive | % | Stimulation | IFN-g responses |
|---|---|---|---|---|---|---|
| Source | Antigen | volunteers | volunteers | assays | Index | (SFCs/1E6 PBMC) |
| Test | 2 | 8 | 100 | 56.25 | 3.3 ± 1.3 | 122.7 ± 90.6 |
| Pf | 5 | 7 | 87.5 | 56.25 | 2.8 + 1.1 | 101.8 ± 74.0 |
| Proteins | 18 | 6 | 75 | 37.5 | 2.2 + 0.2 | 58.4 ± 24.4 |
| | 3 | 6 | 75 | 37.5 | 2.6 + 0.4 | 119.1 ± 69.5 |
| | 22 | 5 | 62.5 | 31.25 | 2.9 ± 0.9 | 108.4 ± 78.1 |
| | 20 | 4 | 50 | 25 | 2.5 ± 0.5 | 74.8 ± 40.1 |
| | 11 | 4 | 50 | 25 | 3.1 ± 0.8 | 81.3 ± 47.9 |
| | 21 | 4 | 50 | 31.25 | 2.3 ± 0.3 | 48.2 ± 16.5 |
| | 13 | 4 | 50 | 31.25 | 2.9 ± 1.1 | 92.2 ± 50.1 |
| | 1 | 3 | 37.5 | 25 | 2.4 ± 0.2 | 61.4 ± 42.0 |
| | 17 | 3 | 37.5 | 18.75 | 2.4 ± 0.3 | 57.6 ± 34.5 |
| | 12 | 3 | 37.5 | 18.75 | 2.2 ± 0.3 | 48.2 ± 16.5 |
| | 16 | 2 | 25 | 12.5 | 2.2 ± 0.3 | 27.2 ± 23.3 |
| | 15 | 2 | 25 | 12.5 | 2.5 ± 0.7 | 28.8 ± 20.0 |
| | 19 | 2 | 25 | 12.5 | 2.7 ± 0.6 | 31.3 ± 22.6 |
| | 9 | 2 | 25 | 12.5 | 2.5 ± 0.2 | 32.0 ± 28.3 |
| | Range | 2-8 | 25-100 | 12.5-56.25 | 2.0-5.9 | 10.0-254.0 |
| | (Mean) | (4.1) | (50.8) | (27.3) | (2.6) | (80.3) |
| Known | CSP | 3 | 37.5 | 18.75 | 2.7 ± 0.6 | 41.6 ± 20.1 |
| Pf | SSP2 | 5 | 62.5 | 31.25 | 2.9 ± 1.0 | 97.2 ± 82.0 |
| Proteins | LSA1 | 2 | 25 | 12.5 | 3.2 ± 1.4 | 34.2 ± 29.0 |
| | EXP1 | 1 | 12.5 | 6.25 | 3.4 | 41.3 |
| | Range | 1-5 | 12.5-62.5 | 6.25-18.75 | 2.0-4.2 | 12.3-230.0 |
| | (Mean) | (2.75) | (34.4) | (17.2) | (2.9) | (65.5) |
| Control | CMV, | 7 | 87.5^{d} | 50.0 | 4.0 ± 2.2 | 59.0 ± 30.5 |
| Epitopes | EBV, | | | | | |

More specifically, in those studies, to identify for analysis a set of ORFs representing antigens potentially expressed in the hepatic stage of the parasite life cycle, MS/MS spectra of peptide sequences generated by multidimensional protein identification technology (hereafter “MudPIT”, Washburn and Yates, 2000) of P. falciparum sporozoite preparations were scanned against the P. falciparum genomic sequence database. A total of 1049 proteins were identified, selecting those ORFs with the highest number of peptide hits. A panel of 27 ORFs representative of putative P. falciparum antigens with a range of expression levels, stage-specificity, and membrane-association were selected. Frequency of recognition in the P. falciparum proteome dataset ranged between 1-16 peptide hits from six different sporozoite runs. CSP, SSP2 and Exp1 were identified during the proteomic analysis, by between 2 (CSP) to 24 (SSP2) peptide hits associated with between 10% (CSP) to 73% (Exp1) sequence coverage. LSA I was not identified since it is expressed only during the hepatic stage and MudPIT studies were not carried out with liver-stage preparations. When searched against the final published P. falciparum database (Gardner et al. 2002) using refined gene model predictions, and taking into consideration genomic sequence information from the Anopheles (vector) (Holt et al. 2002) and human (host) (Venter et al. 2001; Lander et al. 2001) databases, 19 of the 27 antigens were independently identified in either P. falciparum cDNA libraries (P Blair and J Aguiar, unpublished) or as orthologs in P. yoelli laser capture microdissection datasets (J Sacci and J Aguiar).

Amino acid sequences from the 27 ORFs and the four known P. falciparum antigens: circumsporozoite protein (CSP), sporozoite surface protein 2 (SSP2), liver-stage antigen (LSA) and exported proteins I (Exp 1) were scanned with HLA supertype specific algorithms (A1, A2, A3/A 11, B7 and DR supertypes) (Sidney and Sette 1999), and peptide epitopes predicted to bind with the highest affinity to each of the five HLA supertypes were identified (top 10 scorers selected from each supertype/protein combination for a maximum of 50 peptides per protein).

Control peptides from PftSP, SSP2, LSA1 and Exp1 predicted according to the same criteria applied to the 27 ORFs were also synthesized. Analysis revealed that the predictive algorithms were highly effective in identifying the majority (approximately 81%) of previously validated epitopes from those antigens. Peptides representing predicted epitopes were synthesized and then tested by IFN-γ ELIspot for their capacity to induce recall IFN-γ immune responses using PBMC from volunteers immunized with irradiated P. falciparum sporozoites (n=4), or mock immunized control volunteers (n=4). Peptides were tested as pools, at 1 ug/ml each peptide, with each antigen represented by a separate pool, Positive and negative control epitopes from well characterized antigens (CMV, Influenza, EBV, HIV) were also included. A total of 16 of the 27 previously untested antigens were reproducibly recognized as antigens by at least two of eight irradiated sporozoite immunized volunteers tested in two or more assays, but not by any of the four mock immunized controls (Doolan et al. 2003) (Table 1, FIGS. 1A & 1B).

Nine of the 27 antigens were recognized by at least 50% of irradiated sporozoite volunteers in at least 25% of assays and classified as highly antigenic. These antigens could be prioritized for vaccine development accordingly to their immune reactivity (frequency and magnitude of response). Three antigens were recognized by at least 25% of volunteers in at least 15% of assays and classified as intermediately antigenic. Four antigens were recognized by two volunteers and classified as weakly antigenic. Finally, 11 of the 27 untested antigens failed to induce reproducible IFN-γ responses of sufficient magnitude to meet our criteria of positivity. Positive control peptides derived from CMV, FLU and EBV were recognized by 87.5% of the volunteers tested. Pools of predicted epitopes from the known antigens, PfCSP, PfSSP2, PfLSA1 and PfExp1, were recognized by about one third of the irradiated sporozoite volunteers.

The antigens identified as a result of these studies represent, to the best of our knowledge, the first antigens identified from P. falciparum genomic sequence data and shown to be recognized by sporozoite-induced T cell responses. Particularly noteworthy, the immune reactivity against several of the newly identified antigens greatly exceeded the reactivities observed against the well characterized antigens currently considered prime preerythrocytic stage vaccine candidate antigens, namely PfCSP, PfSSP2, PfLSA1 and PfExp1. These data suggest that some of the novel antigens identified using the lmmunosense screening strategy may represent better candidates for vaccine development.

The most antigenic protein amongst those identified by the ImmunoSense approach was a single-exon 549-nucleotide gene (182 amino acid protein) located on P. falciparum chromosome 12 (PFL0800c), and was designated “Ag2”. In immune T cell screening studies (Doolan et al. 2003), Ag2 was reproducibly recognized in the context of multiple genetic restrictions by 100% (8/8) volunteers experimentally immunized with radiation-attenuated P. falciparum sporozoites, but not by any mock-immunized control volunteers, in more than 50% of assays (Table 1).

Moreover, it appeared to be preferentially recognized by irradiated sporozoite-immunized voluteers who were protected against parasite challenge as compared to unprotected volunteers (FIG. 2). Interestingly, responses to the previously characterized antigens (CSP, etc) have been detected predominantly in nonprotected volunteers. In all in vitro studies to date, immune responses to Ag2 have been markedly better than responses to PfExp1, PfLSA1, PfCSP or PfSSP2, as evidenced by the frequency of responders (eg., 8/8 vs. 1/8, 2/8, 3/8 and 5/8; p=0.001, 0.007, and 0.026), and magnitude of response (122.7 vs 41.3, 34.2, 41.6, 97.2) (Doolan et al. 2003) (Table 1). The relative immunodominance of Ag2 as compared to the historic antigens has been consistently demonstrated in subsequent studies.

In independent studies, seven of the 27 ORFs evaluated in the ImmunoSense approach were assayed in a humoral immunoscreening assay designed to evaluate whether putative antigens could be recognized by malaria-immune sera. Of those, 4/4 antigens classified with high immune reactivity and 1/1 antigens classified with intermediate immune reactivity were shown to be recognized by malaria-immune sera but not by malaria-naïve sera, in as many as 5/8 independent experiments (Regis et al. mns submitted). For Ag2, responses that met criteria of positivity were detected in 2 of 3 independent experiments.

### EXAMPLE 2

### Expression Analysis of Ag2

Plasmodium falciparum antigen 2 (PfAg2) appears to be highly expressed specifically in Plasmodium sporozoites based on two large-scale expression analyses. Transcription profiling using an AFFYMETRIX GENE CHIP® (LeRoch et al. 2003) demonstrated PfAg2 to have the 11^{th} highest expression level (5,585.2) overall in sporozoites and falling only behind PftSP (28,684.1) and PfSSP2 (12,475.8) for sporozoite stage-specific genes. Secondly, MudPIT proteomic analysis found Ag2 only matching peptides present in sporozoite stages. Moreover, peptides identified in sporozoites were numerous (SeqCount=17; SpecCount=56) and achieved high sequence coverage (49.5%). These data relatively translate to PfAg2 being in the top four most abundant sporozoite specific antigens via MudPIT analysis. Only PfUSP, PfSSP2, and PFLO945w (hypothetical protein) possessed higher SpecCounts for sporozoite specific antigens.

### EXAMPLE 3

### Identification of Orthologs of PfAg2 in Other Plasmodium spp

No ortholog of PfAg2 could be identified in the annotated P. yoelli genome (Carlton et al. 2002). However, upon scanning the corresponding region along chromosome 12 common stretches of sequence and gene homology was achieved between P. yoelli and P. falciparum. It was noticed that genes flanking Ag2 in the P. falciparum chromosome 12 genome all had orthologs present in P. yoelii. After obtaining the corresponding P. yoelli contig (chrPy1_{—}00049) and scanning for ORFs a previously uncharacterized ORF was identified that shares size (single exon; predicted 185 amino acids for P. yoelii, 182 amino acids for P. falciparum) and sequence identify (44% identity at the amino acid level; 57% identify at the nucleotide level). Subsequently, PfAg2 orthologs were determined in corresponding contigs in P. vivax (185 amino acids; 42% identity at the amino acid level; 54% identify at the nucleotide level) and P. knowlesi (185 amino acids; 43% identity at the amino acid level; 56% identify at the nucleotide level), using BLAST searches against the incomplete sequence databases (Table 2).

TABLE 2
Amino acid and nucleotide sequence identity

| Identity | PfAg2 | PyAg2 | PvAg2 | PkAg2 |
|---|---|---|---|---|
| PfAg2 | — | 57% | 54% | 56% |
| PyAg2 | 44% | — | 64% | 63% |
| PvAg2 | 42% | 55% | — | 89% |
| PkAg2 | 43% | 54% | 85% | — |

DNA
Amino Acid

### EXAMPLE 4

### Sequence Conservation of Ag2 Relative to Well Characterized P. falciparum Antigens

The level of sequence identity between the identified Plasmodium spp orthologs of Ag2 is greater than that'observed between PyHEP17 and PfExp-1 (37% identity at the amino acid level), P. falciparum and P. yoelli circumsporozoite protein (26%) (Dame et al. 1984; Lal et al. 1987); P. falciparum and P. knowlesi circumsporozoite protein (25%); and P. falciparum and P. yoelli sporozoite surface protein (28%) (Robson et al. 1999, 1990; Rogers et al. I 992a, b); and similar to that between the P. falciparum and P. knowlesi sporozoite surface protein 2 (36%). There are no known P. knowlesi or P. vivax orthologs of PyHEP17/PfExp-1, and no known P. yoelii, P. knowlesi or P. vivax orthologs of PfLSA1, so sequence identity between species cannot be assessed in those cases. The level of identity between Plasmodium orthologs of Ag2 is also greater than for leading blood-stage candidate antigens, merozoite surface protein-1 (MAD20, 29%; Wellcome, 30%) and rhoptry antigen (overall identity 4%; best identity 11%). (FIG. 3)

Analysis of the sequence of Ag2 from a number of different P. falciparum strains and isolates revealed a high degree of conservation between P. falciparum strains, and showed that the polymorphism was restricted to a limited number of amino acid variants.

### EXAMPLE 5

### Recognition of Ag2 in the Context of Multiple Genetic Restrictions

Parasite polymorphism of identified targets of CD8+ and CD4+ T cell mediated protective immunity poses a major obstacle to the development of an effective vaccine, since such polymorphism provides a means for the parasite to evade the host immune response (Doolan and Hoffman, 1997). Thus, an ideal vaccine candidate antigen would be highly conserved between Plasmodium strains and species. The high level of sequence identity noted between P. falciparum: strains and between the P. falciparum, P. vivax, P. knowlesi, and P. yoelli orthologs of Ag2 (FIG. 3) support the potential of Ag2 as a good candidate antigen for vaccine development.

Another obstacle to vaccine development is genetic restriction of the host immune response to the CD8+ and CD4+ T cell epitopes that are the targets of T cell mediated protective immunity (Doolan and Hoffman, 1997). Thus, in addition to being conserved between species, an ideal vaccine candidate antigen would contain multiple T cell epitopes recognized in the context of defined panels of HLA supertypes to ensure that the antigen is capable of being recognized by vaccinees of diverse genetic background. Identification of peptide epitopes that bind with high affinity to multiple HLA supertypes is an integral component of the ImmunoSense strategy. PfAg2 contains multiple HLA degenerate HLA-A1, HLA-A2, HLA-A3/A11, HLA-A24 and HLA-B7 degenerate T cell epitopes, and deconvolution of HLA-supertypes used for the initial identification of Ag2 (Doolan et al. 2003) has established the capacity of these epitopes to bind to multiple HLA molecules as assayed by in vitro peptide binding assays, and to be recognized by T cells derived from volunteer immunized with irradiated-sporozoites as assayed by IFN-g ELIspot (Doolan et al. 2003, DL Doolan, unpublished).

Additionally, sera from 20/20 genetically diverse individuals resident in a hyperendemic region of Kenya recognized PfAg2 by western blot analysis of cells transfected with plasmid DNA encoding PfAg2 (data not presented), demonstrating the unrestricted nature of the antibody response to Ag2. Chicken embryo cells infected with recombinant PfAg2 poxvirus or 293 cells infected with recombinant PfAg2 adenovirus were also recognized by sera from malaria-exposed individuals (data not presented).

Studies assessing the immunogenicity of PfAg2 and PyAg2 in vivo support the contention that Ag2 is recognized in the context of multiple genetic elements. In the studies, BALB/C (H-2d), C57BL/6 (H-2b), B10.BR (H-2k), (H-2a) and CD1 (outbred) mice were immunized under various immunization designs. In study 1, mice were immunized with priming and boosting immunization preparations containing plasmid DNA encoding PfAg2 or PyAg2. In study 2, mice were primed with DNA encoding PfAg2 or PyAg2, and boosted with DNA containing PfAg2 or PyAg2, or boosted with a recombinant poxvirus expression system containing PfAg2 or PyAg2. In study 3, mice were primed with either priming preparation containing DNA encoding PfAg2, PyAg2 or PyCSP or a preparation containing recombinant adenovirus expression system containing PyAg 2, PfAg2 or PyCSP. The mice were boosted with preparation containing either a recombinant adenovirus expression system or a recombinant poxvirus expression system, containing PfAg2, PyAg2 or PyCSP. Splenocytes were harvested at 2 weeks post last immunization for assessment of T cell responses by IFN-g ELispot (FIG. 4A) and multiparameter ICS (FIG. 4B).

Sera was harvested at 2-4 weeks post each immunization for assessment of antibody responses by IFAT against P. yoelli or P. falciparum sporozoites. Table 3 shows antibody responses by IFAT against P. yoelli or P. falciparum sporozoites for study 1. Tables 4A & 4B shows antibody responses by IFAT against P. yoelli or P. falciparum sporozoites for study 2, in which sera post last immunization were pooled and assayed in triplicate. IFAT data for Study 3 are shown in Tables 5A & 5B. ELISA against recombinant PfAg2 protein are shown in FIGS. 5A & 5B, in which pooled sera post last immunization were assayed in quadruplicate. Other studies have established that PfAg2 antisera does not recognize blood-stage parasites (liver stage IFAT not done).

TABLE 3
Assessment of antibody responses by IFAT against
P. yoelii or P. falciparum sporozoites.

| | CD-1 | A/J | C57BL/6 | BALB/c | B10.BR |
|---|---|---|---|---|---|
| 1 | 40 | 40 | 80 | 40 | 0 |
| 2 | 160 | 80 | 40 | 80 | 0 |
| 3 | 320 | 320 | 80 | 40 | 0 |
| 4 | 160 | 80 | 20 | 40 | 0 |
| 5 | 40 | 40 | 80 | 160 | 0 |
| 6 | 320 | 80 | 320 | 80 | 0 |
| 7 | 640 | 80 | 1280 | 80 | 0 |
| 8 | 20 | 80 | 40 | 40 | 0 |
| 9 | 40 | 40 | 40 | 0 | 0 |
| 10 | 0 | 40 | 160 | 0 | 0 |
| GeoMean | 109 | 70 | 92 | 62 | 0 |
| Range | 0-640 | 40-320 | 40-1280 | 0-160 | 0 |

(Study 1: DNA/DNA).

TABLE 4 A
Assessment of antibody responses by
IFAT against P. yoelii sporozoites.
P. yoelii sporozoite IFAT

| | Strain | | | | |
|---|---|---|---|---|---|
| Imniunogens | CD-1 | A/J | C57 BL/6 | BALB/c | B10BR |
| Py DNA/DNA | 20 | 80 | 20 | 160 | 20 |
| Py DNA/DNA | 20 | 40 | 40 | 160 | <1/20 |
| Py DNA/DNA | 20 | 40 | 20 | 80 | <1/20 |
| Py DNA/POX | 2560 | 160 | 40 | 40 | 320 |
| Py DNA/POX | 1280 | 320 | 40 | 40 | 160 |
| Py DNA/POX | 1280 | 320 | 20 | 40 | 160 |
| Pf DNA/DNA | 20 | 20 | 20 | 80 | <1/20 |
| Pf DNA/DNA | 20 | <1/20 | 20 | 80 | <1/20 |
| Pf DNA/DNA | 20 | <1/20 | 20 | 80 | <1/20 |
| Pf DNA/POX | 160 | 40 | 20 | 80 | <1/20 |
| Pf DNA/POX | 80 | 40 | 20 | 80 | <1/20 |
| Pf DNA/POX | 80 | 40 | <1/20 | 80 | <1/20 |

(Study 2: DNA/DNA and DNA/POX)

TABLE 4 B
Assessment of antibody responses by
IFAT against P. falciparum sporozoites.
P. falciparum sporozoite IFAT

| | Strain | | | | |
|---|---|---|---|---|---|
| Immunogens | CD-1 | A/J | C57 BL/6 | BALB/c | B10BR |
| Py DNA/DNA | <1/20 | 20 | <1/20 | 640 | 20 |
| Py DNA/DNA | <1/20 | <1/20 | <1/20 | 640 | <1/20 |
| Py DNA/DNA | <1/20 | <1/20 | <1/20 | 640 | <1/20 |
| Py DNA/POX | 20 | 40 | <1/20 | 40 | 40 |
| Py DNA/POX | 20 | 40 | <1/20 | 40 | 40 |
| Py DNA/POX | <1/20 | 40 | <1/20 | 40 | 40 |
| Pf DNA/DNA | <1/20 | 40 | 320 | 40 | 40 |
| Pf DNA/DNA | <1/20 | 40 | 320 | 40 | 40 |
| Pf DNA/DNA | <1/20 | 40 | 320 | 40 | 40 |
| Pf DNA/POX | 160 | 80 | 80 | 160 | 20 |
| Pf DNA/POX | 160 | 80 | 80 | 160 | 20 |
| Pf DNA/POX | 160 | 80 | 80 | 160 | 20 |

(Study 2: DNA/DNA and DNA/POX).

TABLE 5 A
Assessment of antibody responses by IFAT against
P. yoelii and P. falciparum sporozoites pre-boost.
P. yoelii Ag2 & P. falciparum Ag2 Pre-Boost

| GROUP | IMMUNOGENS | GEOMEAN | RANGE |
|---|---|---|---|
| 1 | PyAg2 DNA/POX | 120 | 20-320 |
| 2 | PyAg2 DNA/ADENO | 23 | 20-40 |
| 3 | PyAg2ADENO/POX | 35 | 20-160 |
| 4 | PfAg2 DNA/POX) | 67 | 20-320 |
| 5 | PfAg2 DNA/ADENO | 48 | 20-160 |
| 6 | PfAg2 ADENO/POX | 31 | 20-40 |
| 7 | PyCSP DNA/POX | 80 | 20-320 |
| 8 | PyCSP DNA/ADENO | 36 | 20-160 |
| 9 | PyCSP ADENO/POX) | 40 | 20-160 |
| | PyAg2 DNA preboost | 54 | 20-320 |
| | PfAg2 DNA preboost | 57 | 20-320 |
| | PyCSP DNA preboost | 53 | 20-320 |

(Study 3: DNA/POX and DNA/ADENO)

TABLE 5 B
Assessment of antibody responses by IFAT against
P. yoelii and P. falciparum sporozoites pre-boost.
P. yoelii Ag2 & P. falciparum Ag2 Pre-Challenge (Post-Boost)

| GROUP | IMMUNOGENS | GEOMEAN | RANGE |
|---|---|---|---|
| 1 | Py Ag2DNA/POX | 214 | 80-320 |
| 2 | Py Ag2DNA/ADENO | 135 | 40-320 |
| 3 | Py Ag2 ADENO/POX | 254 | 160-320 |
| 4 | PfAg2 DNA/POX | 135 | 80-320 |
| 5 | PfAg2 DNA/ADENO | 127 | 80-640 |
| 6 | PfAg2 ADENO/POX | 285 | 80-640 |
| 7 | PyCSP DNA/POX | 269 | 20-20480 |
| 8 | PyCSP DNA/ADENO | 320 | 20-20480 |
| 9 | PyCSP ADENO/POX | 285 | 20-2560 |

(Study 3: DNA/POX and DNA/ADENO)

Thus, it is established that plasmid DNA vaccine encoding Ag2 are immunogenic in mice, as demonstrated by the induction of antibodies which recognize P. falciparum sporozoites by IFAT after a single IM dose, and by the induction of T cells which recognize pools of PfAg2 synthetic peptides by IFN-g ELlspot after two or three IM immunizations. T cell responses after one immunization were not assessed. In other related studies, plasmid DNA constructs encoding hypothetical P. falciparum antigens derived from the P. falciparum genome and generated using the GateWay recombinatorial cloning system, were used to immunize mice via GeneGun particle mediated gene delivery (typically a better delivery method for induction of antibody responses than IM immunization), and the antisera has been analyzed for immunoreactivity against different stages of the parasite by IFAT (Aguiar et al., 2004). In those studies, a total of 22% (21/95) of GateWay clones induced parasite-specific antibody responses, but only 3% (3/95) induced antibodies that recognized P. falciparum sporozoite stages (antisera tested after two immunizations). Those genes were not selected based on stage-specific expression and it is possible that only a small percentage may be expressed on the sporozoite. Nonetheless, the capacity of plasmid DNA encoding Ag2 to induce anti-sporozoite antibodies after a single IM immunization attests to the proposed immunogenicity of Ag2 in vivo, consistent with its demonstrated antigenicity in vitro.

### EXAMPLE 6

### Protective Efficacy of Ag2

The capacity of Ag2 to protect against P. yoelli sporozoite challenge was determined in inbred and outbred mice. BALB/C (H-2d), C57BL/6 (H-2b), B10.BR (H-2k), A/J (H-2a) and CD1 (outbred) mice were primed with plasmid DNA encoding PfAg2 or PyAg2, and boosted with recombinant Ag2 poxvirus (Table 6A). Outbred mice were primed with either DNA encoding for PfAg2 or PyAg2 or preparation containing recombinant Ag2 adenovirus expression system and boosted with preparation containing recombinant Ag2 adenovirus expression system or recombinant Ag2 poxvirus expression system. Outbred mice immunized with P. yoelli CSP were evaluated in parallel, as a comparator group. Mice were challenged at 2 weeks post last immunization with infectious P. yoelli sporozoites (100 spz for inbred strains; 200 spz for outbred CD1) and monitored for development of blood-stage parasitemia by Giemsa stained blood smears on days 5-14. Mice immunized with Antigen 2 immunization have shown significant protection against P. yoelli sporozoite, which is comparable to PyCSP.

TABLE 6A
Protective efficacy of Ag2 against P. yoelii sporozoite challenge.

| | | P. yoelii Ag2 DNA/POX | | P. falciparum Ag2 DNA/POX | |
|---|---|---|---|---|---|
| Strain | H-2 | #infect/#challenge | % protection | #infect/#challenge | % protection |
| BALB/c | H-2d | 0/9 | 0 | 1/9 | 11.1 |
| C57BL/6 | H-2b | 1/9 | 11.1 | 0/9 | 0 |
| A/J | H-2a | 1/9 | 11.1 | 0/9 | 0 |
| B10.BR | H-2k | 1/9 | 11.1 | 1/9 | 11.1 |
| CD-1 | outbred | 4/9 | 44.4 | 5/9 | 55.6 |

Mice immunized with DNA and boosted with recombinant poxvirus.

TABLE 6 B
Protective effecacy of Ag2 against P. yoelii sporozoite challenge.

| | | 0 wks | 8 wks | Sterile Protection | |
|---|---|---|---|---|---|
| Group | Strain | Prime | Boost | # protect | % protect |
| 1 | CD-1 | P. yoelii Ag2 DNA | P. yoelii Ag2 POX | 6/14 | 43% |
| 2 | CD-1 | P. yoelii Ag2 DNA | P. yoelii Ag2 Adeno | 7/14 | 50% |
| 3 | CD-1 | P. yoelii Ag2 adeno | P. yoelii Ag2 POX | 9/14 | 64% |
| 4 | CD-1 | P. falciparum Ag2 DNA | P. falciparum Ag2 POX | 5/14 | 36% |
| 5 | CD-1 | P. falciparum Ag2 DNA | P. falciparum Ag2 Adeno | 8/14 | 57% |
| 6 | CD-1 | P. falciparum Ag2 adeno | P. falciparum Ag2 POX | 10/14 | 71% |
| 7 | CD-1 | P. yoelii CSP DNA | P. yoelii CSP POX | 7/14 | 50% |
| 8 | CD-1 | P. yoelii CSP DNA | P. yoelii CSP Adeno | 8/14 | 57% |
| 9 | CD-1 | P. yoelii CSP adeno | P. yoelii CSP POX | 8/14 | 57% |
| 10 | CD-1 | Infectivity control - 200 spz | — | 0/14 | 0% |
| 11 | CD-1 | Infectivity control - 50 spz) | — | 0/7 | 0% |
| 12 | CD-1 | Infectivity control - 10 spz) | — | 1/7 | 14% |
| 13 | CD-1 | Infectivity control - 2 spz) | — | 2/7 | 29% |
| 14 | CD-1 | Infectivity control - 0.4 spz) | — | 3/7 | 43% |

Mice immunized with DNA or recombinant adenovirus and boosted with recombinant poxvirus or recombinant adenovirus.
DNA dose = 100 ug, route = IM
POX dose = 1E7, route = IM
Adeno dose = 1E8, route = IM
Spz chl = 200 spz

In summary, Ag2 is a novel and promising next generation vaccine candidate antigen identified by integrating bioinformatics, genomics, and molecular immunology. It is a single-exon 549 nucleotide gene (182 amino acid protein) located on P. falciparum chromosome 12. It is highly expressed in the P. falciparum sporozoite/liver proteome as evidences by MudPIT of P. falciparum sporozoites and P. falciaprum sporozoite gene transcript profiles. In immune screening studies, Ag2 is recognized by volunteers either experimentally immunized with radiation-attenuated P. falciparum sporozoites or naturally exposed to malaria, and is preferentially recognized by irradiated sporozoite immunized volunteers who were protected against parasite challenge as compared to unprotected volunteers. Significantly, Ag2 has proved to be more immunogenic than all well-characterized P. falciparum antigens currently in clinical evaluation. Overall, an abundance of data implicates Ag2 as an extremely promising candidate antigen for malaria vaccine development.

TABLE 7
Nucleotide and Amino Acid Sequences for Ag 2 Orthologs

| Plasmodium | Nucleotide | Amino Acid | gene IDs |
|---|---|---|---|
| Species | Sequence | Sequence | (from PlasmoDB) |
| P. falciparum | SEQ ID No. 1 | SEQ ID No. 2 | PFL0800c |
| P. vivax | SEQ ID No. 3 | SEQ ID No. 4 | PVX_123510 |
| P. knowlesi | SEQ ID No. 5 | SEQ ID No. 6 | HPVX_123510 |
| P. yoelii | SEQ ID No. 7 | SEQ ID No. 8 | N/A |
| P. berghei | SEQ ID No. 9 | SEQ ID No. 10 | PB000247.02.0 |
| P. chabaudi | SEQ ID No. 11 | SEQ ID No. 12 | PC000314.05.0 |

### EXAMPLE 7

### Enhancement of Immune Protection Against Malaria with Chloroquine

We recruited 15 healthy volunteers between the ages of 18 and 45 years who had no history of malaria or of living in an area in which malaria is endemic in the 6 months before study entry. Only one volunteer had ever been in an endemic area, several years earlier [37].

We randomly assigned the subjects in a double-blind fashion to two study groups: 10 to a vaccine group and 5 to a control group (FIG. 1). The mean (+/−SD) age of the subjects was 22.0 (+/−)1.5 years in the vaccine group and 24.0 (+/−)1.4 years in the control group; seven subjects in the vaccine group were women, as were four subjects in the control group. Chloroquine was provided to all subjects in a standard prophylactic regimen of a loading dose of 300 mg on each of the first 2 days and then 300 mg once a week, starting on day 7, for a total duration of 13 weeks.

While receiving chloroquine, subjects in the vaccine group were exposed on three occasions at monthly intervals to bites of 12 to 15 mosquitoes that had been infected with P. falciparum, for a total exposure of bites from 36 to 45 infected mosquitoes per subject. Control subjects received bites from an equal number of uninfected mosquitoes on the same occasions.

Anopheles slephensi mosquitoes were reared according cording to standard procedures at our insectary. Infected mosquitoes were obtained by feeding on gametocytes of NF54, a chloroquine-sensitive strain of P. falciparum, as described previously. NF54 is genetically homogeneous but has not been formally cloned. Only the technicians who prepared the mosquitoes were aware of their infectivity status, and these staff members had no clinical involvement with the subjects or the investigators. Blood-engorged mosquitoes were dissected to confirm the presence of sporozoites. If necessary, feeding sessions were repeated until precisely the predefined number of infected mosquitoes had fed. However, a single feeding session was sufficient in 49 of 60 instances of immunization or challenge, whereas a second session was required in just 10 instances and a third session in only I instance. On days 6 to 10 after each immunization by mosquito exposure, all subjects were followed on an outpatient basis, and blood was drawn for standard whole-blood counts and daily peripheral blood smears. Any signs and symptoms were recorded by the attending physician as follows: mild events (easily tolerated), moderate events (interferes with normal activity), or severe events (prevents normal activity).

Eight weeks after the last immunization dose and 4 weeks after the discontinuation of chloroquine prophylaxis, all 15 subjects were challenged by exposure to the bites of five mosquitoes that were infected with the homologous NF54 strain of P. falciparum. This period was considered to be sufficient for chloroquine levels to drop below those that might be inhibitory to parasite multiplication. All subjects were checked twice daily on an outpatient basis from day 5 to day 21 for symptoms and signs of malaria, and hematologic tests and peripheral-blood smears were performed.

If results of peripheral-blood testing were positive, subjects were treated with a standard curative combination regimen of 80 mg of artemether and 480 mg of lumefantrine, followed by five identical doses at 8, 24, 36, 48, and 60 hours. The subjects were then followed closely for 3 days. Complete cure was confirmed on the basis of peripheral blood smears. All subjects who continued to have negative results on the peripheral-blood smear from the day of infection until day 21 after the challenge were presumptively treated with artemether-lumefantrine. Hematologic and biochemical measures were determined in routine fashion at the hospital's central clinical laboratory. The use of nucleic acid sequence-based amplification and real-time polymerase-chain-reaction (PCR) assays to determine the densities of P. falciparum parasites have been described previously [15,16]. Chloroquine levels were measured by liquid chromatography [17,18]. Minimum therapeutic concentrations for plasma chloroquine levels maintained by the laboratory were 30 μg per liter.

Venous whole blood was collected in VACUTAINER™ cell-preparation tubes (CPT, Becton Dickinson) before the first immunization and again before the malaria challenge. Plasma was collected and stored at −70° C. Peripheral-blood mononuclear cells were isolated by density gradient centrifugation, frozen in fetal-calf serum containing 10% dimethyl I sulfoxide, and stored in liquid nitrogen. Antibody titers were assessed by enzyme-linked immunosorbent assay (ELISA) and immunofluorescence assay, according to standard protocols. Cellular responses to cryopreserved asexual parasites were assessed by 24-hour in vitro peripheral-blood-stimulation assays, 22 followed by intracellular cytokine staining with the use of a Fix and Perm Kit (Caltag Laboratories) and flow cytometry.

The results of this study are illustrated in FIG. 8. No parasites were observed in the peripheral-blood smears of any of the 10 subjects in the vaccine group after each of the three immunization sessions during chloroquine prophylaxis. However, after the first immunization, a brief submicroscopic parasitemic episode was detected in all vaccinees (FIG. 8A). This result was expected since chloroquine has no effect against either sporozoites, liver-stage parasites, or the early ring forms of the first generation of blood-stage parasites that are caused by merozoites released from mature hepatic schizonts. As illustrated in FIG. 8A, after each of the subsequent two immunizations, a progressively reduced incidence and burden of submicroscopic parasitemia was seen.

As illustrated in FIG. 8B, after challenge with the homologous NF54 strain of P. falciparum, asexual blood-stage parasites were detected in peripheral-blood smears of all five control subjects between days 7 and 11 after exposure (mean prepatent period, 9.2 days). Real-time PCR analyses revealed the expected cyclical multiplication of blood-stage parasites. Also illustrated in FIG. 8B, there was no evidence of blood-stage parasites in any of the vaccinees at any time during the post-challenge follow-up period until day 21, either by repeated microscopy of peripheral-blood smears or by real-time PCR analysis.

Illustrated in FIG. 9 is the cellular immune responses were assessed by counting cytokine-producing cells in peripheral bloodspecimens from the subjects, with the use of intracellular cytokine staining and flow cytometry after 24 hours of in vitro stimulation with erythrocytes infected with the homologous strain of P. falciparum or with ininfected erythrocytes.

As shown in FIG. 9, although there was no significant difference in the overall proportion of cells producing individual cytokines (interferon-γ or TNF-α) in either group after immunization (day C-1), a significant increase was observed in the proportion of cells producing multiple cytokines in response to infected erythrocytes in vaccinees, as compared with baseline: P=0.03 for the within-group comparison for interferon-γ and interleukin-2; P=0.046 for TNF-α and interleukin-2; and P=0.03 for interferon-?, TNF-α, and interleukin-2 (FIGS. 9A, 9B, and 9C). The importance of these pluripotent lymphocytes in acquired immune protection is suggested by their higher cytokine content and may reflect better effector function (FIGS. 9D, 9E, and 9F). The major contributors to this increase in pluripotent lymphocytes with a response to infected erythrocytes were CD3+CD45RO+ memorylike T cells (P=0.02 for the comparison with day I-1) (FIG. 9G). CD4+CD8− cells showed a particularly marked response (P=0.005 for the comparison with day I-1) (FIG. 9H). Most noticeably, these new pluripotent lymphocytes were predominantly of the effector memory D62L+CD45RO+ phenotype (P=0.005 for the comparison with day I-1), although there was also a small but significant increase in the numbers of responding central memory CD62L+CD45RO+ cells in vaccines (P=0.02 for the comparison with day I-1) (FIG. 9I).

### EXAMPLE 2

### Prophetic Example of Immunization with Concomitant Administration of Chloroquine

A preferred embodiment is a method for inducing an immune response to malaria that comprises the co-administration of an immunogenic composition with an immune modulating drug. In a preferred embodiment, the preferred drug is chloroquine.

In one embodiment, the immunogenic method comprises co-administering an immune modulating drug, such as chloroquine, to a mammal with an immunogenic composition comprising a DNA vector capable of expressing and containing a DNA sequence encoding one or more DNA sequences encoding one or more polypeptides derived from Plasmodium falciparum. The immunogenic composition and chloroquine are administered to the mammal one or more times. In the preferred embodiment, one or more malaria polypeptides may be used alone or in combination including antigen 2(Ag2), apical membrane 1 (AMA1) or circumsporozoite protein (CSP). The expression vectors in the embodiment, includes adenovirus.

### REFERENCES

1. Doolan D L, Dobano C, Baird J K: Acquired immunity to malaria. Clin Microb Reviews 2009, 22:13-36. Comprehensive review on the development of immunity against malaria under natural and experimental conditions.
2. Beeson J G, Osier F H A, Engwerda C R: Recent insights into humoral and cellular immune responses against malaria. Trends Parasitol 2008, 24:578-584.
3. Pombo D J, Lawrence G, Hirunpetcharat C, Rzepczyk C, Bryden M, Cloonan N, Anderson K, Mahakunkijcharoen Y, Martin L B, Wilson D et al: Immunity to malaria after administration of ultra-low doses of red cells infected with Plasmodium falciparum. Lancet 2002, 360:610-617.
4. Hoffman S L, Goh L M L, Luke T C, Schneider I, Le T P, Doolan D L, Sacci J, de la Vega P, Dowler M, Paul C et al: Protection of humans against malaria by immunization with radiation-attenuated Plasmodium falciparum sporozoites. J Infect Dis 2002, 185:1155-1164.
5. Roestenberg M, McCall M, Hopman J, Wiersma J, Luty A J F, van Gemert G J, van de Vegte-Bolmer M, van Schaijk B, Teelen K, Arens T et al: Protection against a malaria challenge by sporozoite inoculation. N Engl J Med 2009, 361:468-477.
6. Epstein J E, Giersing B, Mullen G, Moorthy V, Richie T L: Malaria vaccines: Are we getting closer? Curr Opin Mol Ther 2007, 91:12-24.
7. Abdulla S, Oberholzer R, Juma O, Kubhoja S, Machera F, Membi C, Omari S, Urassa A, Mshinda H, Jumanne A et al: Safety and immunogenicity of RTS, S/ASO2D malaria vaccine in infants. N Engl J Med 2008, 359:2533-2544.
8. Breman J G, Plowe C V: A Malaria Vaccine for Control: More Progress. J. Infect Dis 2009, 200:317-320.
9. Targett G, Greenwood B: Malaria vaccines and their potential role in the elimination of malaria. Malar J 2008, 7:S10.
10. McCall M B B, Netea M G, Hermsen C C, Jansen T, Jacobs L, Golenbock D, van der Ven AJAM, Sauerwein R W: Plasmodium falciparum infection causes proinflammatory priming of human TLR responses. J Immunol 2007, 179:162-171.
11. Coban C, Igari Y, Yagi M, Reimer T, Koyama S, Aoshi T, Ohata K, Tsukui T, Takeshita F, Sakurai K et al: Immunogenicity of Whole-Parasite Vaccines against Plasmodium falciparum Involves Malarial Hemozoin and Host TLR9. Cell Host Microbe 2010, 7:50-61.
12. Griffith J W, Sun T, McIntosh M T, Bucala R: Pure Hemozoin Is Inflammatory In Vivo and Activates the NALP3 Inflammasome via Release of Uric Acid. J Immunol 2009, 183:5208-5220.
13. Mestas J, Hughes C C W: Of mice and not men: differences between mouse and human immunology. J Immunol 2004, 172:2731-2738.
14. Finney O C, Riley E M, Walther M: Regulatory T cells in malaria-friend or foe? Trends Immunol 2010, 31:63-70.
15. D'Ombrain M C, Robinson L J, Stanisic D I, Taraika J, Bernard N, Michon P, Mueller I, Schofield L: Association of Early Interferon-Production with Immunity to Clinical Malaria: A Longitudinal Study among Papua New Guinean Children. Clin Infect Dis 2008, 47:1380-1387.
16. Casares S, Richie T L: Immune evasion by malaria parasites: a challenge for vaccine development. Curr Opin Immunol 2009, 21:321-330. A comprehensive review of the evasion mechanism by malaria parasites manipulating the immune system.
17. Wykes M N, Good M F: What really happens to dendritic cells during malaria? Nat Rev Microbiol 2008, 6:864-870.
18. Ocana-Morgner C, Mota M M, Rodriguez A: Malaria blood stage suppression of liver stage immunity by dendritic cells. J Exp Med 2003, 197:143-151.
19. Wilson N S, Behrens G M, Lundie R J, Smith C M, Waithman J, Young L, Forehan S P, Mount A, Steptoe R J, Shortman K D et al: Systemic activation of dendritic cells by Toll-like receptor ligands or malaria infection impairs crosspresentation and antiviral immunity. Nat Immunol 2006, 7:165-172.
20. Wang R, Arevalo-Herrera M, Gardner M, Bonelo A, Carlton J, Gomez A, Vera O, Soto L, Vergara J, Bidwell S et al: Immune responses to Plasmodium vivax preerythrocytic stage antigens in naturally exposed Duffy-negative humans: a potential model for identification of liver-stage antigens. Eur J Immunol 2005, 356:1859-1868.
21. Aidoo M, Udhayakumar V: Field studies of cytotoxic T lymphocytes in malaria infections: implications for malaria vaccine development. Parasitol Today 2000, 16:50-56.
22. Bejon P, Mwacharo J, Kai O, Todryk S, Keating S, Lowe B, Lang T, Mwangi T W, Gilbert S C, Peshu N et al: The induction and persistence of T cell IFN-□ responses after vaccination or natural exposure is suppressed by Plasmodium falciparum. J Immunol 2007, 179:4193-4201.
23. Belnoue E, Costa F, Frankenberg T, Vigario A M, Voza T, Leroy N, Rodrigues M M, Landau I, Snounou G, Renia L: Protective T cell immunity against malaria liver stage after vaccination with live sporozoites under chloroquine treatment. J Immunol 2004, 172:2487-2495.
24. Aly A S I, Downie M J, Mamoun C B, Kappe S H I: Subpatent infection with Nucleoside Transporter 1-deficient Plasmodium blood stage parasites , confers sterile protection against lethal malaria in mice. Cell Microbiol 2010, doi:10.1111/j.1462-5822.2010.01441.x.
25. Hill A V, Reyes-Sandoval A, O'Hara G, Ewer K, Lawrie A, Goodman A, Nicosia A, Folgori A, Colloca S, Cortese Ret al: Prime-boost vectored malaria vaccines: Progress and prospects. Hum vaccin 2010, 6:78-83.
26. Limbach K J, Richie I L: Viral vectors in malaria vaccine development. Parasite Immunol 2009, 31:501-519.
27. Ballou W R: The development of the RTS, S malaria vaccine candidate: challenges and lessons. Parasite Immunol 2009, 31:492-500.
28. Stewart V, McGrath S M, Dubois P M, Pau M G, Mettens P, Shott J, Cobb M, Burge J R, Larson D, Ware L A et al: Priming with an adenovirus 35-circumsporozoite protein (CS) vaccine followed by RTS, S/AS01B boosting significantly improves immunogenicity to Plasmodium falciparum CS compared to that with either malaria vaccine alone. Infect Immun 2007, 75: 2283-2290.
29. Vaughan A M, Wang R, Kappe S H: Genetically engineered, attenuated wholecell vaccine approaches for malaria. Hum vaccines 2010, 6:107-113.
30. Garulli B, Stillitano M G, Barnaba V, Castrucci M R: Primary CD8+ T-Cell Response to Soluble Ovalbumin Is Improved by Chloroquine Treatment In Vivo. Clin Vaccine Immunol 2008, 15:1497-1504.
31. Accapezzato D, Visco V, Francavilla V, Molette C, Donato T, Paroli M, Mondelli M U, Doria M, Torrisi M R, Barnaba V: Chloroquine enhances human CD8+ T cell responses against soluble antigens in vivo. J. Exp Med 2005, 202:817-828.
32. Belizaire R, Unanue E R: Targeting proteins to distinct subcellular compartments reveals unique requirements for MHC class I and H presentation. Proc Natl Acad Sci USA 2009, 106:17463-17468. Illustration of different endosomal pathways for antigen processing with particular consequences for routing and presentation. Differential effects by application of chloroquine.
33. Nowak A K, Lake R A, Robinson B W S: Combined chemoimmunotherapy of solid tumours: Improving vaccines? Adv Drug Deliv Rev 2006, 58:975-990.
34. Melief C J M: Cancer immunotherapy by dendritic cells. Immunity 2008, 29:372-383.
35. Walther M, Jeffries D, Finney O C, Njie M, Ebonyi A, Deininger S, Lawrence E, Ngwa-Amambua A, Jayasooriya S, Cheeseman I H et al: Distinct Roles for FOXP3+ and FOXP3−CD4+ T Cells in Regulating Cellular Immunity to Uncomplicated and Severe Plasmodium falciparum Malaria. PLoS Pathog 2009, 5:e1000364. Discussion of the relationship between Tregs and clinical presentation. Evidence for inverse correlation between Tregs and malaria-specific memory responses.
36. Coban C, Ishii K J, Kawai T, Hemmi H, Sato S, Uematsu S, Yamamoto M, Takeuchi O, Itagaki S, Kumar N el al: Toll-like receptor 9 mediates innate immune activation by the malaria pigment hemozoin. J Exp Med 2005, 201:19.
37.Roestenberg, M., M. McCall, J. Hopman, J. Wiersma, A. Luty, G. Gemert, M van de Vegte-Bolmer, B. Van Schaijk, K. Teelen, T. Arens, L. Spaarman, Q. de Mast, W. Roeffen, G. Snounou, L. Renia, A. van der Ven, C. Hermsen, and R. Sauerwein, Protection against a malaria challenge by sporozoite inoculation. NEJM 2009, 361: 468-477.
38. Aguiar, J. C. et al., (2004).” High throughput generation of P. falciparum functional molecules by recombinational cloning” Genome Research. In Press.
39. Allen, T. M., et al. (2000). “Induction of AIDS virus-specific CTL activity in fresh, unstimulated peripheral blood lymphocytes from rhesus macaques vaccinated with a DNA prime/modified vaccinia virus Ankara boost regimen.” J Immunol 164(9): 4968-78.
40. Amara, R. R., et al. (2001) Control of a mucosal challenge and prevention of AIDS by a multiprotein DNA/MVA vaccine. Science. 292(5514):69-74.
41. Barouch, D. H., et al. (2001a). Elicitation of high-frequency cytotoxic T-lymphocyte responses against both dominant and subdominant simian-human immunodeficiency virus epitopes by DNA vaccination of rhesus monkeys. J Virol. 2001 March; 75(5):2462-7.
42. Barouch, D. H., et al. (2001b). Reduction of simian-human immunodeficiency virus 89.6P viremia in rhesus monkeys by recombinant modified vaccinia virus Ankara vaccination. J Virol. 75(11):5151-8.
43. Beadle, C. and S. L. Hoffman (1993). “History of malaria in the United States Naval Forces at war: World War I through the Vietnam conflict.” Clin. Infect. Dis. 16: 320-329.
44. Bender, B. S., Rowe, C. A., Taylor, S. F., Wyatt, L. S., Moss B., and Small P. A. Jr. (1996). Oral immunization with a replication-deficient recombinant vaccinia virus protects mice against influenza. J Virol. 70(9):6418-24.
45. Berglund, P., et al. (1998). “Enhancing immune responses using suicidal DNA vaccines.” Nat Biotechnol 16(6): 562-5.
46. Berglund, P., et al. (1999). “Immunization with recombinant Semliki Forest virus induces protection against influenza challenge in mice.” Vaccine 17(5): 497-507.
47. Carlton, J. M., et al. (2002). “Genome sequence and comparative analysis of the model rodent malaria parasite Plasmodium yoelii yoelii.” Nature 419: 520-526.
48. Dame, J. B., et al. (1984). “Structure of the gene encoding the immunodominant surface antigen on the sporozoite of the human malaria parasite Plasmodium falciparum.” Science 225: 593-599.
49. Doolan, D. L., et al. (1996a). “Characterization of the protective Hepatocyte Erythrocyte
Protein 17 kDa gene of Plasmodium yoelii: homolog of P. falciparum Exported Protein-1.” J. Biol. Chem. 271: 17861-17868.
50. Doolan, D. L., et al. (1996b). “Circumventing genetic restriction of protection against malaria with multi-gene DNA immunization: CD8⁺ T cell, interferon-□, nitric oxide dependent immunity.” J. Exp. Med. 183: 1739-1746.
51. Doolan, D. L. and S. L. Hoffman (1997). “Multi-gene vaccination against malaria: a multi-stage, multi-immune response approach.” Parasitol. Today 13: 171-178.
52. Doolan, D. L. and S. L. Hoffman (1999). “IL-12 and NK cells are required for antigen-specific adaptive immunity against malaria initiated by CD8⁺ T cells in the Plasmodium yoelii model.” J Immunol 163: 884-892.
53. Good, M. F. and D. L. Doolan (1999). “Immune effector mechanisms in malaria.” Curr Opin Immunol 11: 412-419.
54. Doolan, D. L. and S. L. Hoffman (2000). “The complexity of protective immunity against liver-stage malaria.” J Immunol 165: 1453-1462.
55. Doolan, D. L. and S. L. Hoffman (2001). “DNA-based vaccines against malaria: status and promise of the Multi-Stage Malaria DNA Vaccine Operation.” Int J Parasitol 31(8): 753-62.
56. Doolan, D. L., et al. (2003). “Identification of new P. falciparum antigens by antigenic analysis of genomic and proteomic data.” Proc Natl Acad Sci USA 100(17): 9952-9957.
57. Eloit, M., et al., Construction of a defective adenovirus vector expressing the pseudorabies virus glycoprotein gp50 and its use as a live vaccine. J Gen Virol, 1990. 71(Pt 10): p. 2425-31.
58. Epstein, J. E., et al. (2002). “Safety, Tolerability, and Lack of Antibody Responses After Administration of a PfCSP DNA Malaria Vaccine via Needle or Needle-Free Jet Injection, and Comparison of Intramuscular and Combination Intramuscular/Intradermal Routes.” Hum Gene Ther 13(13): 1551-60.
59. Florens, L., M. P. Washburn, et al. (2002). “A proteomic view of the Plasmodium falciparum life cycle.” Nature 419: 520-526.
60. Gardner, M. J., N. Hall, et al. (2002). “Genome sequence of the human malaria parasite Plasmodium falciparum.” Nature 419(6906): 498-511.
61. Gardner, M. J., et al. (2002). “Genome sequence of the human malaria parasite Plasmodium falciparum.” Nature 419(6906): 498-511.
62. Hevey, M., et al., Marburg virus vaccines based upon alphavirus replicons protect guinea pigs and nonhuman primates. Virology, 1998. 251(1): p. 28-37.
63. Hirsch, V. M., Fuerst, T. R., Sutter, G., Carroll, M. W., Yang, L., Goldstein, S., Piatak, M., Elkins, W. R., Alvord, W. G., Montefiori, D. C., Moss, B., and Lifson, J. D. (1996). Patterns of viral replication correlate with outcome in simian immunodeficiency virus (SIV)-infected macaques: effect of prior immunization with a trivalent SIV vaccine in modified vaccinia virus Ankara. J. Virol. 70: 3741-3752.
64. Hoffman, S. L. and Doolan, D. L. (2000). “Malaria Vaccines—Targeting the infected hepatocyte.” Nature Med. 6: 1218-1219.
65. Holt, R. A., G. M. Subramanian, et al. (2002). “The genome sequence of the malaria mosquito Anopheles gambiae.” Science 298(5591): 129-49.
66. Kariu, T., Ishino, T., Yano, K., Chinzei, Y, and Yuda, M. (2006). “CelTOS, a novel malarial protein that mediates transmission to mosquito and vertebrate hosts.” Molecular Micro. 59(5):1369-1379.
67. Klonjkowski B, et al. (1999). Adenoviral vectors for vaccines., In Adenoviruses: Basic biology to gene therapy, S. P, Editor. RG Landers Company: Austin. p. 163-173.
68. Kumar, S., et al. (2002). “A multilateral effort to develop DNA vaccines against falciparum malaria.” Trends Parasitol 18(3): 129-35.
69. Lal, A. A., et al. (1987). “Structure of the gene encoding the circumsporozoite protein of Plasmodium yoeli.” J. Biol. Chem. 262: 2937-2940.
70. Lander, E. S., et al. (2001). “Initial sequencing and analysis of the human genome.” Nature 409(6822): 860-921.
71. Lasonder, E., et al. (2002). “Analysis of the Plasmodium falciparum proteome by high-accuracy mass spectrometry.” Nature 419(6906): 537-42.
72. Le Roch, K. G., et al. (2003). “Discovery of gene function by expression profiling of the malaria parasite life cycle.” Science 301(5639): 1503-8.
73. Le, T. P., et al. (2000). “Safety, tolerability and humoral immune responses after intramuscular administration of a malaria DNA vaccine to healthy adult volunteers.” Vaccine 18(18): 1893-1901.
74. Leitner, W., et al. (2000). Enhancement of tumor-specific immune response with plasmid DNA replicon vectors. Cancer Res. 60:51.
75. Martin, T., et al. (1999). “Plasmid DNA malaria vaccine: the potential for genomic integration following intramuscular injection.” Human Gene Ther 10: 759-768.
76. Mayr, G. A., J. Chinsangaram, and M. J. Grubman. (1999). Development of replication-defective adenovirus serotype 5 containing the capsid and 3C protease coding regions of foot-and-mouth disease virus as a vaccine candidate. Virology. 263(2): p. 496-506.
77. Moss B. Genetically engineered poxviruses for recombinant gene expression, vaccination, and safety. Proc Natl Acad Sci USA. 1996 Oct. 15; 93(21):11341-8.
78. Moss, B., Carroll, M. W., Wyatt, L., Bennink, J. R., Hirsch, V. M., Goldstein, S., Elkins, W., Fuerst, T., Lifson, J., Piatak, M., Restifo, N., Overwijk, W., Chamberlain, R., Rosenberg, S. A., and Sutter, G. (1996). Host range restricted, non-replicating vaccinia virus vectors as vaccine candidates. Adv. Exp. Med. Biol. 397: 7-13.
79. Nardin, E., F. Zavala, et al. (1999). “Pre-erythrocytic malaria vaccine: mechanisms of protective immunity and human vaccine trials.” Parassitologia 41(1-3): 397-402.
80. Ourmanov, I., Brown, C. R., Moss, B., Carroll, M., Wyatt, L., Pletneva, L., Goldstein, S., Venzon, D., and Vm., H. (2000). Comparative efficacy of recombinant modified vaccinia virus Ankara expressing simian immunodeficiency virus (SIV) Gag-Pol and/or Env in macaques challenged with pathogenic SIV. J. Virol. Methods 74: 2740-2751.
81. Parker, S. E., F. Borellini, et al. (1999). “Plasmid DNA malaria vaccine: tissue distribution and safety studies in mice and rabbits.” Hum. Gene Ther. 10: 741-758.
82. Parker, S. E., D. Monteith, et al. (2001). “Safety of a GM-CSF adjuvant-plasmid DNA malaria vaccine.” Gene Ther 8(13): 1011-23.
83. Plebanski, M. and A. V. Hill (2000). “The immunology of malaria infection.” Curr Opin Immunol 12(4): 437-41.
84. Polo et al. (1999). Stable alphavirus packaging cell lines for Sindbis virus and Semliki Forest virus-derived vectors. Proc. Natl. Acad. Sci. USA 96:4598.
85. Pushko, P., et al. (2000). Recombinant RNA replicons derived from attenuated Venezuelan equine encephalitis virus protect guinea pigs and mice from Ebola hemorrhagic fever virus. Vaccine, 2000. 19(1): p. 142-53.
86. Pushko, P., et al., Replicon-helper systems from attenuated Venezuelan equine encephalitis virus: expression of heterologous genes in vitro and immunization against heterologous pathogens in vivo. Virology, 1997. 239(2): p. 389-401.
87. Richie, T. L. and A. Saul (2002). “Progress and challenges for malaria vaccines.” Nature 415(6872): 694-701.
88. Robson, K. J., et al. (1988). “A highly conserved amino-acid sequence in thrombospondin, properdin and in proteins from sporozoites and blood stages of a human malaria parasite.” Nature 335: 79-82.
89. Robson, K. J., et al. (1990). “Polymorphism of the TRAP gene of Plasmodium falciparum.” Proc. R. Soc. Lond. B. Biol. Sci. 242: 205-216.
90. Rogers, W. O., et al. (1992). “Characterization of Plasmodium falciparum sporozoite surface protein 2.” Proc. Natl. Acad. Sci. USA 89: 9176-9180.
91. Rogers, W. O., et al. (1992). “Characterization of the gene encoding sporozoite surface protein 2, a protective Plasmodium yoelii sporozoite antigen.” Mol. Biochem. Parasitol. 53: 45-52.
92. Sacci, J. B., Aguiar, J. C., Lau, A. O., Hoffman, S. L., (2002) “Schneider, J., Gilbert, S. C., Hannan, C. M., Degano, P., Prieur, E., Sheu, E. G., Plebanski, M., and Hill, A. V. (1999). Induction of CD8+ T cells using heterologous prime-boost immunisation strategies. Immunol. Rev. 170: 29-38.
93. Sedegah, M., et al. (2002). “Persistence of protective immunity to malaria induced by DNA priming and poxvirus boosting: characterization of effector and memory CD8(+)-T-cell populations.” Infect Immun 70(7): 3493-9.
94. Sedegah, M., et al. (1994). “Protection against malaria by immunization with plasmid DNA encoding circumsporozoite protein.” Proc. Natl. Acad. Sci. USA 91: 9866-9870.
95. Sedegah, M., et al. (1998). “Boosting with recombinant vaccinia increases immunogenicity and protective efficacy of malaria DNA vaccine.” Proc Natl Acad Sci USA 95(13): 7648-53.
96. Sedegah, M., et al. (2000). “Improving protective immunity induced by DNA-based immunization: priming with antigen and GM-CSF-encoding plasmid DNA and boosting with antigen-expressing recombinant poxvirus.” J Immunol 164(11): 5905-12.
97. Sette, A. and J. Sidney (1999). “Nine major HLA class I supertypes account for the vast preponderance of HLA-A and -B polymorphism.” Immunogenetics 50: 201-212.
98. Shiver, J. W., et al. (2002). Replication-incompetent adenoviral vaccine vector elicits effective anti-immunodeficiency-virus immunity. Nature. 415(6869):331-335.
99. Srivastava, I. K. and M. A. Liu (2003). “Gene vaccines.” Ann Intern Med 138(7): 550-9.
100. Sullivan, N. J., Sanchez, A., Rollin, P. E., Yang, Z. Y., Nabel, G. J. (2000). Development of a preventive vaccine for Ebola virus infection in primates. Nature. 408(6812):605-609.
101. Sutter, G., Wyatt, L. S., Foley, P. L., Bennink, J. R., and Moss, B. (1994). A recombinant vector derived from the host range-restricted and highly attenuated MVA strain of vaccinia virus stimulates protective immunity in mice to influenza virus. Vaccine 12: 1032-1040.
102. Venter, J. C., et al. (2001). “The sequence of the human genome.” Science 291(5507): 1304-51.
103. Wang, R., D. L. Doolan, et al. (1998). “Malaria DNA vaccine induces antigen specific genetically restricted cytotoxic T lymphocytes in humans.” Science 282: 476-480.
104. Wang, R., et al. (2001). “Induction of CD4(+) T cell-dependent CD8(+) type 1 responses in humans by a malaria DNA vaccine.” Proc Natl Acad Sci USA 98(19): 10817-22.
105. Washburn, M. P. and J. R. Yates, 3rd (2000). “Analysis of the microbial proteome.” Curr Opin Microbiol 3(3): 292-7.
106. Yarosh, O. K., et al., (1996). Human adenovirus type 5 vectors expressing rabies glycoprotein. Vaccine, 14(13): p. 1257-64.
107. Ying, H., et al. (1999). “Cancer therapy using a self-replicating RNA vaccine.” Nat Med 5(7): 823-827.

What is claimed is:

1. A method to induce protective immunity comprising:
a. administrating a priming immunization preparation containing a plasmodium antigen or a fragment thereof; and
b. administrating a boosting immunization preparation containing said plasmodium antigen or fragment thereof.

2. The method of claim 1, wherein said plasmodium antigen is selected from a group consisting of PfAg2, PyAg2, PvAg2, PkAg2, PoAg2, PmAg2, PbAg2, PcAg2 and a combination thereof.

3. The method of claim 1, wherein said priming immunization preparation containing said antigen or fragment thereof, is selected from a group consisting of:
a. a recombinant virus expression system;
b. a recombinant protein antigen or a recombinant polypeptide;
c. a synthetic peptide;
d. a DNA vector;
e. a whole organism or extract thereof; and
f. a combination thereof.

4. The method of claim 1, wherein said boosting immunization preparation containing said antigen or fragment thereof, is selected from a group consisting of
a. a recombinant virus expression system;
b. a recombinant protein antigen or a recombinant polypeptide;
c. synthetic peptide;
d. a DNA vector;
e. a whole organism or extract thereof; and
f. a combination thereof.

5. The method of claim 3 or claim 4, wherein said recombinant virus expression system comprises plasmodium antigen 2, fragment thereof, or epitope derived from said antigen, or combination thereof.

6. The method of claim 3 or claim 4, wherein said recombinant virus expression system is selected from the group consisting of poxvirus, adenovirus, adeno-associated virus, and retrovirus.

7. The method of claim 5, wherein said poxvirus is selected from the group consisting of cowpox, canarypox, monkeypox, and fowlpox.

8. The method of claim 1, wherein said antigen is native or recombinant.

9. The method of claim 1, wherein the number of doses of priming agent is 1-4 and the number of doses of boosting agent is 1-4.

10. The method of claim 1, wherein said immunization preparations are administered by routes selected from the group consisting of subcutaneous, intramuscular, intradermal, mucosal, oral, transcutaneous, and by injection devices, or combinations thereof.

11. The method of claim 1, wherein said immunization preparation further comprising one or more co-administration of an immune modulating drug.

12. The method of claim 11, wherein said immune modulating drug is chloroquine or cyclophosphamide.

13. A vaccine comprising an immunogenic preparation containing a plasmodium antigen or fragment thereof, wherein said antigen is selected from a group consisting of PyAg2, PfAg2, PvAg2, PkAg2, PoAg2, PmAg2 and a combination thereof.

14. The vaccine of claim 14, further comprising an immune modulating drug.

15. The vaccine of claim 14, wherein said immune modulating drug is chloroquine or cyclophosphamide.

16. A method to enhance protective immunity against malaria comprising:
a. administering an antigen capable of inducing a protective immunity against malaria; and
b. administering an immune modulating drug.

17. The method of 16, wherein said immune modulating drug is chloroquine or cyclophosphamide.


SEQUENCE LISTING (12 sequences)

SEQ ID NO: 1 - DNA, 549 bp, Plasmodium falciparum
atgaatgccttaagaagattaccagttatttgctctttcttagtatttcttgtcttttccaatgttttatgtttcagaggaaacaacggacacaattcttcatcatctctctataatggaagccaatttattgaacaattaaataacagttttacttcagcttttcttgaatcacaatcaatgaataagattggtgatgatttagcagagaccatatcaaatgaacttgtcagtgttttacaaaaaaattcaccaacctttttagaatcaagctttgatatcaaatcagaagtaaaaaaacacgcaaaatctatgttaaaggaattaatcaaagtaggattgccatcattcgaaaatctcgtagctgaaaatgttaaaccaccaaaagtcgacccagcaacatatggtataatagtaccagtattaacatctttatttaataaggtagaaacagctgtaggtgcgaaagtttctgatgagatatggaattacaattcaccagacgtctcagaaagtgaagaaagtttatcagatgattttttcgattaa

SEQ ID NO: 2 - protein, 182 aa, Plasmodium falciparum
MNALRRLPVICSFLVFLVFSNVLCFRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFD

SEQ ID NO: 3 - DNA, 558 bp, Plasmodium vivax
atgaacaaagtaaaccgagtctcgattatttgtgctttcttggcacttttttgcttcgtaaatgtgttgtccttgcggggaaagagcggctcgactgcctcgtcttctcttgaaggaggaagcgaattttccgagcgcatagggaacagcttatcgtcattcctttccgaatcagcatctttggaagttattggaaatgaactggccgacaacatcgccaacgaaattgttagctccctgcaaaaggattcagcatcctttttacaaagtgggtttgacgtaaaaacccagttaaaggctactgccaagaaggtcttagtggaagcgttaaaagcagcattagagccaacggaaaaaattgttgcctccacgattaagccaccacgtgtcagcgaagatgcctacttcttattgggaccggtcgtcaagactctctttaacaaagttgaggacgttttacacaagccaatacctgataccatttgggaatacgaatccaagggttccctcgaagaggaagaagctgaagatgagttctctgatgagttgttagattag

SEQ ID NO: 4 - protein, 185 aa, Plasmodium vivax
MNKVNRVSIICAFLALFCFVNVLSLRGKSGSTASSSLEGGSEFSERIGNSLSSFLSESASLEVIGNELADNIANEIVSSLQKDSASFLQSGFDVKTQLKATAKKVLVEALKAALEPTEKIVASTIKPPRVSEDAYFLLGPVVKTLFNKVEDVLHKPIPDTIWEYESKGSLEEEEAEDEFSDELLD

SEQ ID NO: 5 - DNA, 558 bp, Plasmodium knowlesi
atgaacaaagtaaatcgagtctcgattatttgtgctttcttagcacttttttgcttcgtaaatgtgttgtccttgaggggaaagagcggattgactgcttcgtcttctcttgaaggaggaagcgaattttccgagcgcatagggaacaccttatcgtcgttcctttcagaatcggcgtccttggaagtaattggaaatgaattggccgacaacatcgccaacgaaattgttggctccctgcaaaacgattcagcatcctttttacaaagtgagtttgacgtaaaagcccagttaaaagctactgccaagaaggtcttaacggaagcattaaaagcagcgttagagccaacggaaaaaattgttgcctctacgattaagccaccacgtatcaaggaagatatctactttttattgagcccggtcgtaaggtctctctttaacaaagttgaggacgttttacataagccagtatctgatgacatttggaattacgaatcaaggggttcctcctccgaggaagaagatgaagttgattcagatgaagatttcttagattaa

SEQ ID NO: 6 - protein, 185 aa, Plasmodium knowlesi
MNKVNRVSIICAFLALFCFVNVLSLRGKSGLTASSSLEGGSEFSERIGNTLSSFLSESASLEVIGNELADNIANEIVGSLQNDSASFLQSEFDVKAQLKATAKKVLTEALKAALEPTEKIVASTIKPPRIKEDIYFLLSPVVRSLFNKVEDVLHKPVSDDIWNYESRGSSSEEEDEVDSDEDFLD

SEQ ID NO: 7 - DNA, 558 bp, Plasmodium yoelii
atgaataaattaacaaaattgtcagttatatcatctgtccttgtatttttttgcttttttaatgttctatgtttgagaggcaaaaatggatcagaaatgtcgaaatttcttgaaggtggagtcgaatcttccaatcgtataaaaaacagtttgtcgtcatttatatccgaatctgcatcacttgatgatattggaaatggattagccgaaacaattaccaatgaaatctttagtgcatttcaacaagactcatcatcatttttgcaaacccaatttgacataaaaaaacacataaaagaaaatgcaaagaaagtacttattgaagccataagattaggattggagccagttgagaaaattgtagcaaaatctattcaacctcctaaggtcaaccgtcatacatactcattggtgtcaccaatagtaaaagcgctttttaacaagatagaagacgccgttcacaagccagttaatgataatatatgggaatatgaaggtggtgatgaagaatatgacgaaaatgaagaagaaaacttcgataatgatttctttaattaa

SEQ ID NO: 8 - protein, 558 aa, Plasmodium yoelii
ATGAATAAATTAACAAAATTGTCAGTTATATCATCTGTCTTTGTATTTTTTTGCTTTTTTAATGTTCTATGTTTGAGAGGCAAAAATGGATCAGAAATGTCGTCATTTCTTGAAGGTGGAGTCGAATCTTCCAATCGTATAAAAAAAAGTTTAGCGTCATTTATATCCGAATCTTCATCACTTGATGATATTGGAAATGGATTAGCTGAAACAATTACCAATGAAATATTTAGTGCATTTCAACAAGACTCGTCATCATTTTTGCAAACCAAATTTGACATAAAAAAACATATAAAAGAAAATGCAAAGAAAGTACTTATTGAAGCCATAAGATTAGGATTGGAGCCAGTTGAGAAAATTGTAGCACAATCTATTCAACCTCCTAAAGTCAACCGCCATACATACTCATTGGTGTCCCCAGTAGTAAAAGCACTTTTTAACAAGATAGAAGAAGCCGTTCACAAGCCAGTTAGTGATAATATATGGGACTATGCAGGGGGTGATGATGAATATGAAGAAACTGAAGAAGACAACTTCGATAATGATTTCTTTAATTAA

SEQ ID NO: 9 - DNA, 558 bp, Plasmodium berghei
atgaataaattaacaaaattgtcagttatatcatctgtctttgtatttttttgcttttttaatgttctatgtttgagaggcaaaaatggatcagaaatgtcgtcatttcttgaaggtggagtcgaatcttccaatcgtataaaaaaaagtttagcgtcatttatatccgaatcttcatcacttgatgatattggaaatggattagctgaaacaattaccaatgaaatatttagtgcatttcaacaagactcgtcatcatttttgcaaaccaaatttgacataaaaaaacatataaaagaaaatgcaaagaaagtacttattgaagccataagattaggattggagccagttgagaaaattgtagcacaatctattcaacctcctaaagtcaaccgccatacatactcattggtgtccccagtagtaaaagcactttttaacaagatagaagaagccgttcacaagccagttagtgataatatatgggactatgcagggggtgatgatgaatatgaagaaactgaagaagacaacttcgataatgatttctttaattaa

SEQ ID NO: 10 - protein, 185 aa, Plasmodium berghei
MNKLTKLSVISSVFVFFCFFNVLCLRGKNGSEMSSFLEGGVESSNRIKKSLASFISESSSLDDIGNGLAETITNEIFSAFQQDSSSFLQTKFDIKKHIKENAKKVLIEAIRLGLEPVEKIVAQSIQPPKVNRHTYSLVSPVVKALFNKIEEAVHKPVSDNIWDYAGGDDEYEETEEDNFDNDFFN

SEQ ID NO: 11 - DNA, 555 bp, Plasmodium chabaudi
atgaataaattaacaaaattgtcggttatatcatctgtccttgtatttttttgcttttttaacgttctttgtttgagaggcaaaaatgggtcagaaatgtcatcatcacttgaaggtggagtggaatctgccattcgtataaaaaacagtttagagtcatttatatcagaatctgcatcattggatgatattggaaatggattagctgaaacaattaccaatgaaatctttagtgcagttcaacaagactcatcatcatttttacaaactcaatttgacataaaaaaacatataaaagaaaatgcaaagaaagtacttattgaagccataagattaggattggaaccagttgagaagattgtagcaaaatctattcaaccccctaaaatcaaccgacatacatactcattagtgtctccagtagtaaaatcactttttaacaagatagaagaggctgttcacaaaccagttagtgatagtatatgggagtatgaaggtggtgaagaatacgacgaaagtgaagaagataactacgaggatgagttatttaattaa

SEQ ID NO: 12 - protein, 184 aa, Plasmodium chabaudi
MNKLTKLSVISSVLVFFCFFNVLCLRGKNGSEMSSSLEGGVESAIRIKNSLESFISESASLDDIGNGLAETITNEIFSAVQQDSSSFLQTQFDIKKHIKENAKKVLIEAIRLGLEPVEKIVAKSIQPPKINRHTYSLVSPVVKSLFNKIEEAVHKPVSDSIWEYEGGEEYDESEEDNYEDELFN